(19) United States
(12) Patent Application Publication
(10) Pub. No.: US 2020/0218874 A1
(43) Pub. Date: Jul. 9, 2020

(54) A LABEL-FREE METHOD AND SYSTEM FOR MEASURING DRUG RESPONSE KINETICS OF THREE-DIMENSIONAL CELLULAR STRUCTURES

(71) Applicant: AGENCY FOR SCIENCE, TECHNOLOGY AND RESEARCH, Singapore (SG)

(72) Inventors: Lie Yong Judice Koh, Singapore (SG); Ramanuj Dasgupta, Singapore (SG); Giridharan Periyasamy, Singapore (SG)

(21) Appl. No.: 16/638,551

(22) PCT Filed: Aug. 14, 2018
(86) PCT No.: PCT/SG2018/050412

§ 371 Date: Feb. 12, 2020

(30) Foreign Application Priority Data
Aug. 14, 2017 (SG) 10201706639T

(51) Int. Cl. G06K 9/00; C12N 5/09; G01N 33/50; G06K 9/62; G06N 20/00; G06N 5/04

(52) U.S. Cl. CPC G06K 9/0014; C12N 5/0693; G01N 33/5011; G06K 9/6269; G06K 9/00214; G06K 2209/05; G06N 20/00; G06N 5/04; C12N 2503/02; G01N 2500/10; G06K 9/6259

(57) ABSTRACT

Disclosed herein are methods of providing a computational model for predicting an activity of a test agent with respect to a 3D cell structure. Also disclosed herein are label-free prediction methods and a device configured to perform the methods as disclosed herein.

### CROSS-REFERENCE TO RELATED APPLICATIONS

This application claims the benefit of priority of SG provisional application No. 10201706639T, filed 14 Aug. 2017, the contents of it being hereby incorporated by reference in its entirety for all purposes.

### FIELD OF INVENTION

The present invention relates generally to the field of image processing, bioinformatics and cell biology. In particular, the present invention relates to the use of image processing for measuring drug response kinetics in three-dimensional cellular structures.

### BACKGROUND

Unlike monolayer two-dimensional (2D) cell cultures, three-dimensional (3D) tumour spheroid models recapitulate the spatial microenvironment and potentially mimic the pathophysiological responses of the primary tumours. However, most cell-based assays for evaluation of cell viability and drug efficacy are still based on monolayer 2D cell cultures that have not proven to be adequately predictive of in-vivo efficacy. With the emergence of precision oncology, in-vivo like models are increasingly important for the investigation of therapeutic options, and this has spanned a new wave of interests in establishing 3D tumour spheroids as an alternative or complementary screening model for drug testing. In fact, several studies reveal that compared to corresponding 2-D cell cultures of the same cell line, the genomic and proteomic profiles of 3D spheroids are more reflective of the cell-cell interaction and microenvironment of the parental tumours. Published data has also shown that extracellular matrix (ECM) and hypoxia components are significantly elevated in the spheroids, suggesting that 3D models are more appropriate for studies in metastasis and differentiation. Additionally, the efficacy of some drugs is highly dependent on the cell-cell interactions in 3D microenvironment and therefore can be artificially suppressed in 2D cell cultures.

Cancer tumour spheroids have been used in various aspects of cancer research for decades. Various form of tumour spheroids have been established, including multi-cellular tumour spheroids, tumorospheres such as mammospheres, colonospheres, and tissue-derived tumour spheres, and organotypic multi-cellular spheroids. However, it is only in recent years that developments in HCS and HTCS, along with advancement in microscopy technology had made possible the establishment of large-scale 3D tumour spheroid screening platform for therapeutic evaluation. On such 3D HCS/HCTS platforms, thousands of spheroids can be generated in a single experiment and interrogated in parallel with diverse compound libraries and at different dilution. Morphological changes presented by the spheroids can be captured using high-resolution microscopy and assessed or quantified using computational image analysis pipelines. The ability to screen 3D tumour spheroids in a high-throughput manner is critical to its use in rapid drug testing, particularly for personalized therapeutic evaluation in precision oncology.

For example, a well-formed spheroid of at least 500 μm exhibits a concentric structure with distinct proliferating, quiescent and necrotic zones, and a pathophysiological gradient that closely mimic the concentration depreciation of nutrients, oxygen, and metabolites from blood vessels in an in-vivo tumour. Compared to monolayer cell cultures, tumour spheroids generally show reduced physiochemical response to chemotherapeutic drugs. Many treatments with high efficacy in 2D cell cultures have diminished inhibition activity in 3D tumour spheroids, possibly due to the regulation of tumour microenvironment physiology established by the cell-cell and cell-matrix interactions in a 3D settings and a drug penetration gradient established by the presence of a hypoxic necrotic zone, a quiescent zone that is inherently drug-resistant, and the proliferating zone that is directly exposed to the effect of the drugs.

The 3D tumour spheroids have proven to be a prevailing tool for therapeutic evaluation, both in the negative and positive selection of drugs. As an in-vivo like model that better recapitulates the primary tumours, tumour spheroid models can be used for high-throughput chemical screening (HCTS) to enable elimination of false positives (of 2D monolayer models) and thereby reduce down-stream animal testing. Recent studies have also revealed that certain signalling pathways are only activated in a 3D context due to factors such as the presence of cell-matrix interactions. Hence, the tumour spheroid models can be used to identify drugs which may have an in-vivo efficacy but whose activity is suppressed in a 2D monolayer models. When tumour spheroids are cultured from the cell-lines derived from a patient, the tumour spheroids model patient specific factors that can alter the response of drug due to metabolic and micro-environmental differences, and can be a valuable tool for precision oncology studies.

A major hurdle in using 3D tumour spheroids as a drug-screening model is the lack of automated methods for measuring drug response kinetics in spheroids. Non image-based assays have been developed to determine cell viability or cytotoxicity. These include the use of ATP to quantify metabolically active cells, or resazurin reduction to quantify mitochondria metabolic activity, 4-nitrophenyl phosphate to measure cytosolic acidic phosphastase (APH) levels, and tetrazolium salt to measure Lactate dehydrogenase (LDH) activity. The measurements are mostly based on absorption, luminescence, or fluorescence. Image-based assays typically involve live/dead cell staining and acquiring the images through multiple channels. The need for cell fixation in both non image- and image-based assays restrict the assays to end point experiments and limit the ability to monitor pharmacodynamics of the spheroids in response to drug treatments.

On the other hand, morphological-based methods primarily use the size/volume or shape of the spheroids. However, these parameters do not adequately reflect phenotypic responses. Comparatively, zone-specific image descriptors are more predictive of the pharmacological response of the tumour spheroids but with limited predictive value (R<0.5). More complex image descriptors will be required to attain more accurate quantification of the drug response at each time-point, and to profile the kinetics of the drug response over time.

Thus, what is needed is a label-free, non-invasive system can be established for continuous monitoring of the response kinetics of the 3D spheroids in a high-throughput set-up. Furthermore, other desirable features and characteristics will become apparent from the subsequent detailed description and the appended claims, taken in conjunction with the accompanying drawings and this background of the disclosure.

### SUMMARY

In one aspect, the present invention refers to A method of providing a computational model for predicting an activity of a test agent with respect to a 3D cell structure, the method comprising providing a plurality of training samples, each training sample comprising a respective training test agent of a plurality of training test agents applied to a respective training 3D cell structure of a plurality of training 3D cell structures; determining a respective image of at least one training sample of the plurality of training samples; determining a respective set of features of each of the respective images; determining a respective activity of the respective training test agent applied to the respective training 3D cell structure corresponding to at least one training sample of the plurality of training samples; determining the computational model based on the determined respective sets of features and the determined respective activities.

In another aspect, the present invention refers to A label-free prediction method comprising: providing a computational model; providing a sample comprising a test agent applied to a 3D cell structure; determining an image of the sample; determining a set of features of the image; predicting an activity of the test agent with respect to the 3D cell structure based on the set of features and based on the computational model.

In yet another aspect, the present invention refers to a device configured to perform the method as disclosed herein.

### FIGURES

FIG. 1 shows a schematic and a micrograph of the concentric structure of a tumour spheroid derived from the lymph node biopsy of a head and neck cancer patient. The spheroid presented a proliferation gradient with diminishing oxygen and nutrients content from outer rind of the spheroid to the inner hypoxic core and distinct proliferating, quiescent and necrotic zones.

FIG. 2 shows micrograph images of spheroids derived from a head and neck cancer patient were treated with high (a) and low (b) concentration of anticancer drug—Cisplatin, and without treatment in DMSO media (c). All three patient-derived spheroids are of similar sizes of approximately 500 microns. However, at low concentration of Cisplatin, the morphological structure of the tumor spheroid resembled that of the untreated spheroid in DMSO media while the spheroid treated with higher concentration of Cisplatin showed an enlarged core zone (enclosed in yellow) reflective of the efficacy of the treatment.

FIG. 3 shows a brightfield image of spheroids grown in an ultra-low attachment 384 well plate. Each well contained a tumour spheroid treated with one of 480 anti-cancer drugs.

FIG. 4 shows the results of a Pearson correlation analysis of 504 image features, which were extracted from the segmented images of 1,170 drug treated spheroids and correlated to the drug response (y axis; based on CellTiter-Glo® 3D Cell Viability Assay).

FIG. 5 shows micrograph images of a head and neck tumour spheroid was segmented into the Proliferating (bottom row, left frame), Quiescent (bottom row, middle frame) and Necrotic (bottom row, right frame) zones. Image features were then independently quantified from the three zone-specific images in the lower panel.

FIG. 6 shows an example of an image segmentation workflow through the “Spheroid Peeling method”. A number of different methods can be used to perform object segmentation. Given that spheroid peeling is based on brightfield images, segmentation is conducted in the brightfield channel where the images are represented as pixels with different intensity levels. In Cell Profiler, identification of primary object is achieved using (1) thresholding and (2) filtering. The thresholding step involves identifying the foreground region from the background region using Maximum correlation threshold (Padmanabhan K, Eddy W F, Crowley J C (2010) “A novel algorithm for optimal Image thresholding of biological data” Journal of Neuroscience Methods 193, 380-384). Simply, the MCT method determines the threshold by minimizing the variance within each region. The filtering step then involves refining the boundary of the objects through splitting objects (declumping) or merging objects. The method used is Laplacian of Gaussian (R. Haralick and L. Shapiro, Computer and Robot Vision, Vol. 1, Addison-Wesley Publishing Company, 1992, pp 346-351). The Laplacian is a measure of the second spatial derivative of an image. The Laplacian of an image will enhance regions of rapid intensity change, that is edges, and hence it can be used for edge detection.

FIG. 7 shows a schematic of a machine learning workflow for generating a quantitative model of drug response in tumour spheroids using over 600K zone-specific image features generated from 1,231 tumour spheroid images. The learned model can be used to quantify the drug response of the spheroids across all time-points.

FIG. 8 shows the results of a regression analysis (left) and a box plot (right), showing comparative high correlation of r=0.77 between Label-Free Oncology Score (LaFOS) and drug response, as well as a general increase of drug response indicated via the LaFOS over time.

FIG. 9 shows the results of a 4-dimensional drug response of tumor spheroids to 3 anti-cancer compounds. Duplicate spheroids were used to assess each drug (R1 and R2). The spheroids of the patient showed increasing response to NVP-TAE684 and GSK2126458 over the course of 72 hours and hitting an inhibition rate of 60% and 80% respectively, while they are unresponsive to BEZ235.

FIG. 10 shows a dot plot showing the correlation between the drug response and 491 image features.

FIG. 11 shows a schematic of the overall methodology of the approach disclosed herein. Biopsies of the patients were obtained and derived into cell lines for screening purposes. The experimental phase involved establishing high-throughput experimental pipelines to (i) generate tumour spheroids from cell lines, (ii) screen tumour spheroids with drug libraries of small molecule inhibitors, and (iii) acquire high-resolution confocal microscope images of the spheroids at regular time intervals. The second aim involved development of computational technologies enabling a 4D HCS system, including methods to (i) reconstruct a “3D image” from multiple z-plane micrographs of the spheroids, (ii) generate a multi-parametric machine learning model to predict drug response from the morphological changes of spheroids over time, and (iii) derive 4D drug response kinetics (phenomics profile) from (iv) and stratify them to select candidate drugs for the patient.

FIG. 12 shows the overall procedure with respect to, for example, patient samples. (A) shows a tumour from a head and neck cancer patient. (B) shows a well-formed spheroid showing distinct proliferating, quiescent and necrotic zones. Drug sensitivity in tumour spheroids manifest in form of morphological changes, with the drug sensitive spheroid exhibiting larger necrotic core. (C) shows an image of the tumour spheroids cultured in 384-well ultra-low attachment plates. (D) shows micrograph images of tumour spheroids in the presence of YM155 (a drug with known effect in the inspected cell line), and in control DMSO. (E) shows a correlation matrix of the genome-wide gene expression profiles of 3D tumour spheroids/PDMTs, 2D monolayer cultures, primary tumour and PDX of the same patient reveals highly correlated transcriptomic profiles between the spheroid model and the primary tumour. (F) shows a table of the pathway enrichment analysis of genes with elevated expression in the 3D tumour spheroids (compared to the monolayer cell cultures) suggests that 3D tumour spheroid models shown an enrichment in KRAS signalling, ECM organisation and cancer stem cells, which are reflective of tumourigenesis and metastasis.

FIG. 13 shows a Pearson correlation graph and a linear correlation graph, depicting the optimizing of the machine learning method in LaFOS to improve the accuracy of the prediction. The latest results show that the LaFOS of the tumour spheroids show a significantly higher correlation with corresponding drug efficacy scores (R=0.81, RMSE=13.2), an improvement from the method as previously disclosed.

FIG. 14 shows a schematic outlining the reasoning behind the development of the method disclosed herein.

FIG. 15 shows line graphs depicting the stratification of drug evaluation based on their response kinetics.

FIG. 16 shows results from the first preliminary test conducted in relation to the invention of LaFOS. The image of a patient-derived tumour spheroid in DMSO (control) is segmented into proliferating (red), quiescent (yellow) and necrotic zones (green).

FIG. 17 shows the results from the first preliminary test conducted in relation to the invention of LaFOS. The image of a patient-derived tumour spheroid in compound YM155 is segmented into proliferating (red), quiescent (yellow) and necrotic zones (green).

FIG. 18 shows the results from the first preliminary test conducted in relation to the invention of LaFOS. The image of a patient-derived tumour spheroid in compound Gefitinib is segmented into proliferating (red), quiescent (yellow) and necrotic zones (green).

FIG. 19 shows the first part of a schematic of a machine learning workflow for generating a quantitative model of drug response in tumour spheroids using over 600K zone-specific image features generated from 1,231 tumour spheroid images. In this section, the schematic shows how the high-resolution images are used to arrive at a drug response model.

FIG. 20 shows the second part of a schematic of a machine learning workflow for generating a quantitative model of drug response in tumour spheroids using over 600K zone-specific image features generated from 1,231 tumour spheroid images. Here, it is shown how data from multi-well experiments can be used to result in a drug response prediction.

FIG. 21 shows micrograph images of spheroids. The method as disclosed herein had been applied to 11 cell-lines from 4 indications—7 Head and neck (HN120M, HN120P, HN137M, HN137P, HN148M, HN160P and HN182M), 1 Breast (MDA-MB-231), 1 Ovarian (OV169AP) and 2 Colorectal cancer (CRC948 and HCT116). They include 9 patient-derived lines and 2 commercial cell-lines—MDA-MB-231 and HCT116. The tumour spheroids generated from these cell-lines were independently screened with the Selleck Anti-cancer and Kinase Inhibitor small molecule libraries, comprising more than 600 compounds. The tumour spheroids generated from these cell-lines were imaged at 5 time-points—0 hours, 24 hours, 48 hours, 72 hours and 96 hours (or 120 hours). Shown here are example images of the spheroids derived from the 11 cell-lines at 72 hours. These images were segmented into the necrotic, quiescent and proliferating zones, and the efficacies of the drugs were predicted using Deep learning.

FIG. 22 shows line graphs of the drug response kinetics of the top inhibitors from 6 cancer cell lines, which are the top inhibitors identified for five (5) head and neck cancer, and one (1) ovarian cancer cell-line, using the method disclosed herein. The drug response scores were predicted from the morphological patterns of the spheroid at 24 hours, 48 hours, 72 hours and 96 hours (or 120 hours). Specifically, the efficacies of YM155 in the HN137M and Flavopiridol in HN120M were already validated in in vivo models and previous published in an independent study (R. Haralick and L. Shapiro, Computer and Robot Vision, Vol. 1, Addison-Wesley Publishing Company, 1992, pp 346-351; see FIG. 23).

FIG. 23 shows line graphs depicting the in vivo validation experiments of Flavopiridol in HN120M and YM155 in HN137M. In the right figure, six independent cohorts of mice (n=6) bearing patient-matched PDX in one flank were treated with vehicle (control) and 5 mg kg⁻¹ Flavopiridol (HN120). In the left figure, five independent cohorts of male mice (n=5), bearing tumours on both flanks from HN137P PDX and HN137M PDX, were treated with 2 mg kg⁻¹ of YM155, compared to vehicle (control). A significant anti-tumour effect was observed for YM155 treatment in HN137M PDX while HN137P PDX did not display significant sensitivity to YM155. Two-tail Student's t test was carried out; *P<0.05, **P<0.01, ***P<0.001.

### DETAILED DESCRIPTION

The present disclosure describes a method and system to measure the response kinetics of 3-dimensional (3D) cellular structures, for example tumour spheroids, in presence of drug or drug combinations. By way of an example, upon drug treatment, 3D tumour spheroids exhibit zone-specific morphological changes that can be captured using high spatial resolution bright-field microscopy. These morphological changes can be accurately quantified using complex computational image descriptors. The method disclosed herein exploits the zone-specific morphological changes over time to determine the response kinetics of the tumour spheroid to a given drug/drug combination, and/or the pharmacokinetics of such a drug/test agent in the 3D cellular structure. As the method disclosed herein utilizes, among others, bright-field images, and does not require fixing or staining of the spheroids, a label-free, non-invasive system can be established based on the disclose herein for continuous and dynamic monitoring of the response kinetics of, for example, the 3D spheroids in presence of different environmental cues. Furthermore, the method in accordance with the present disclosure utilizes machine-learning methods to generate multivariate models of image features with improved predictivity of the drug response.

As used herein, the term “response kinetics”, also known as “pharmacodynamics”, refers to the biological and/or chemical response of the 3D cellular structure, for example a spheroid as disclosed herein, to the presence of the test agent. Such response kinetics can include, but are not limited to, parameters such as, cell morphology, overall structural changes, adherence or the lack thereof, anchoring of the cells to the vessel wall, necrosis or cell death, changes in cell surface markers, changes in environmental pH levels within the culture vessel and the like.

As used herein, the term “pharmacokinetics” refers determining the fate of substances administered to a living organism. In the present disclosure, the term pharmacokinetics refers to the effect of the 3D cellular structure on the test agents. In other words, the study of pharmacokinetics concerns itself with the metabolism of the cellular structure and the resulting metabolites of the one or more test agents. Taken together, the information gained through pharmacodynamic and pharmacokinetic analyses can be used to determine treatment parameters, for example, but not limited to, dosage ranges, dosage regimes, adverse effects, side effects and drug benefits.

As used herein, the phrase “label-free prediction method” refers to a process of prediction or detection that does not require an additional step of labelling either the test agent or the target cells (i.e. 3D cell structure) with any labelling process. In some examples, the label-free prediction method may be performed without the need to optically stain either the 3D cell structure or the test agent. In some examples, the label-free prediction method does not require the step of covalently attaching a fluorophore or other reporter molecule to either the test agent or the 3D cell structure.

There are multiple translational applications of the subject matter disclosed herein. The method disclosed herein can be implemented in a large-scale 3D high throughput chemical screening (HTCS) and/or high content screening (HCS) drug testing platform to enable parallel interrogation of cellular structures, including but not limited to tumour spheroids, with over hundreds of drug or drug combinations and at different dilution levels, thus enabling ranking and selection of therapeutic options.

This 3D drug-testing platform can be used, for example, as a pre-animal testing step to determine the pharmacodynamics and pro-longed effect of a candidate drug such as a standard-of-care chemotherapy on tumour spheroids derived from cancer patients. The tumour spheroids can be cultured, for example, from immortalized cell-lines or primary cell-lines of patients. In the latter case, the system enables a comprehensive assessment of the response kinetics of different therapeutic options for individual patients and provides enhanced information for therapeutic selection, thus facilitating personalized oncology and precision therapy.

Thus, in one example, a sample as disclosed herein is obtained from a diseased subject. In one example, the subject has cancer. In another example, the sample is cultured into a three-dimensional structure. Such a three-dimensional structure can be, but is not limited to, spheroids (also referred to as spherical structures), globular structures and the like. As used herein, the term “spheroid” refers to a “sphere-like” structure. In contrast, the term “globular” refers to a globe-like structure, which can include sphere-like structures as well as structures comprised of multiple globes. A flattened sphere, for example, would be considered to fall under the term “globular”, but would not count as being spherical. Also encompassed are organoid models, which are similar to spheroids in shape, spherical organoids, as well as spherical, organoidal 3D cellular structures. Samples disclosed herein can be, for example, obtained from solid or liquid biopsy samples. The samples obtained herein can also be clinical or samples from naturally occurring tissues. In another example, samples can comprise tumour cells.

Once obtained from a subject, such samples can be grown in cell culture, either under adherent or low-attachment or non-adherent conditions, in order to obtain spheroid cellular structures according to methods known in the art. In one example the 3D structure disclosed herein comprises tumour cells. In another example, a spheroid as disclosed herein comprises tumour cells.

The method disclosed herein involves segregation of areas of the cell spheroids into three distinctive zones (necrotic, quiescent and proliferating) and the construction of multi-variate drug response models using multiple image features extracted from each zone. While the presence of these zones is commonly known in the art and had been previously discussed, individual image features had been associated with drug response but not as a multi-variate model.

Thus, in one example, the spheroid comprises a necrotic zone, a quiescent zone and a proliferating zone. In another example, the zones of the spheroid comprise a necrotic zone, a quiescent zone and a proliferating zone. In another example, the spheroid comprises a quiescent zone and a proliferating zone. In one example, it is possible that only two zones from a spheroid can be computationally segmented—in such a case, these two zones would be the quiescent and proliferating zones. The necrotic core cannot firmly establish itself when there is still supply of oxygen and nutrients to the centre of the spheroids. Therefore, in such cases, it would only be possible to determine the presence of two zones. In cases where the spheroids do not exhibit any zonal differentiation, the method as disclosed herein cannot be applied. This happens, for example, when spheroids are not entirely formed and/or a loose aggregation of cells is present.

As used herein the term “quiescence”, in reference to a quiescent zone, quiescence refers to cells, or a zone or region of a 3D in which the cells are dormant with minimal basal activity. In other words, a quiescent zone comprises cells that are viable but do not proliferate.

The spheroids disclosed herein comprise of cells, based on which a person skilled in the art will appreciate that the zones, once defined, can be circular or irregular, for example, showing up as a band around a certain area of the spheroid, or even as a defined section of the spheroid. Such zones do not necessarily encompass the spheroid, but may also be found as a region of cells of the spheroid located to once side of the same. In another example, the method as disclosed herein comprises determining the size or width of each zone and comparing them to the respective base line measurements. It is understood that changes in the zone sizes are indicative of whether a tested agent is considered to be effective or not effective in the treatment of said 3D cellular structure. In other words, changes in zone sizing are indicative of the efficacy of the drug in treatment and/or the response of the 3D cellular structure to the drug.

Methods known in the art had previously evaluated drug response as a function of intensity gradient of the core zone to the periphery of the tumour spheroid. Another method known in the art discussed a method to segregate the tumour spheroids into three overlapping areas—core, halo and periphery in invasion assays. The method disclosed herein defines three distinctive zones that do not correspond in their entirety to those zones as determined using methods known in the art. Also known methods in the art had typically focused on the density gradient across the three areas of the tumour spheroids, while the method disclosed herein extracts multiple features from each zone of the spheroids.

Thus, disclosed herein is a method wherein machine learning is applied to associate morphological changes in tumour spheroids to drug response. More specifically, the machine learning is applied to determine a computational model to associate morphological changes in tumour spheroids in response to drug treatment. The determining of the computational model includes training the computational model and determining parameters of the computational model. Thereafter, in accordance with the present disclosure, the computational model is utilized to output an activity or response score of a test agent or drug with respect to a 3D cell structure. For example, the computational model is configured to output an inhibition score of the test agent or drug with respect to the 3D cell structure. In one example, cell cytotoxicity can be used to filter out toxic compounds, and thus can be used in conjunction with cell viability for therapeutic drug selection.

Given that the method disclosed herein can be applied to brightfield images, the cellular structures disclosed herein need not be fixed. That is to say that the cellular structures disclosed herein do not need to be anchored or adhered to the surface of a reaction vessel in order to be analysed, nor do the cells need to be chemically halted in their present state, thus enabling continuous monitoring of the morphological changes, for example, in the spheroid zones and corresponding predictions of drug response in a temporal manner. This enables, for example, continuous profiling of the response kinetics of each tumour spheroid over time simply through high-resolution microscopy imaging.

Unlike monolayer 2-dimensional (2D) cell cultures, 3D tumour spheroid models recapitulate the spatial microenvironment and mimic the pathophysiological responses of the primary tumours. However, most cell-based assays for evaluation of cell viability and drug efficacy are still based on monolayer 2D cell cultures that have not proven to be adequately predictive of in-vivo efficacy. With the emergence of precision oncology, in-vivo like models are increasingly important for the investigation of therapeutic options, and this has spanned a new wave of interests in establishing 3D tumour spheroids as an alternative or complementary screening model for drug testing. In fact, several studies reveal that compared to corresponding 2-D cell cultures of the same cell line, the genomic and proteomic profiles of 3D spheroids are more reflective of the cell-cell interaction and microenvironment of the parental tumours. It has been shown that extracellular matrix (ECM) and hypoxia components are significantly elevated in the spheroids, indicating that 3D models are more appropriate for studies in metastasis and differentiation. Additionally, the efficacy of some drugs is highly dependent on the cell-cell interactions in 3D microenvironment and therefore can be artificially suppressed in 2D cell cultures.

Cancer tumour spheroids have been used in various aspects of cancer research for decades. Examples of various forms of (tumour) spheroids have been established, including multi-cellular tumour spheroids, tumourospheres such as mammospheres, colonospheres, and tissue-derived tumour spheres, and organotypic multi-cellular spheroids. Multi-cellular tumour spheroids are developed by re-aggregating cells in cell cultures in non-adherent condition. Examples of tumorospheres, which include mammospheres (if the spheres are composed of breast cancer cells) and colonospheres (for spheres comprising of colon cancer cells), can be developed from the proliferation of cancer stem/progenitor cells and grown in serum-free medium supplemented with growth factors. Tissue-derived tumour spheres can be obtained, for example, from partially dissociating tumour tissue and re-compacting the cells into a spherical structure. Organotypic multi-cellular spheroids can be developed by cutting tumour tissues and rounding the tissues in non-adherent condition. Thus, in one example, the 3D cellular structure can be, but is not limited to, spheroid, organoid, or tumoursphere. However, it is only in recent years that developments in high content screening and high throughput chemical screening, along with advancement in microscopy technology had potentiated the establishment of large-scale 3D tumour spheroid screening platform for therapeutic evaluation. On such 3D platforms, thousands of spheroids can be generated in a single experiment and interrogated in parallel with diverse compound libraries and at different dilution. Morphological changes presented by the spheroids can be captured using high-resolution microscopy, and assessed or quantified using computational image analysis pipelines. The ability to screen 3D tumour spheroids in a high-throughput manner is critical to its use in rapid drug testing, particularly for personalized therapeutic evaluation in precision oncology.

Thus, in one example, the method disclosed herein can be utilised in high content screening and/or high throughput chemical screening. Such screenings can be manual or automated.

The size of a 3D cellular structure has an average diameter of between 100 μm to 1000 μm, or at least 100 μm, or at least 200 μm, or at least 300 μm, or at least 400 μm, or at least 410 μm, or at least 420 μm, or at least 430 μm, or at least 440 μm, or at least 450 μm, or at least 460 μm, or at least 470 μm, or at least 480 μm, or at least 490 μm, or at least 500 μm, or at least 510 μm, or at least 520 μm, or at least 530 μm, or at least 540 μm, or at least 550 μm, or at least 560 μm, or at least 570 μm, or at least 580 μm, or at least 590 μm, or at least 600 μm, or at least 700 μm, or at least 800 μm, or at least 900 μm. In one example, the 3D cellular structure has an average diameter of about 500 μm. In another example, the spheroid has an average diameter about 500 μm.

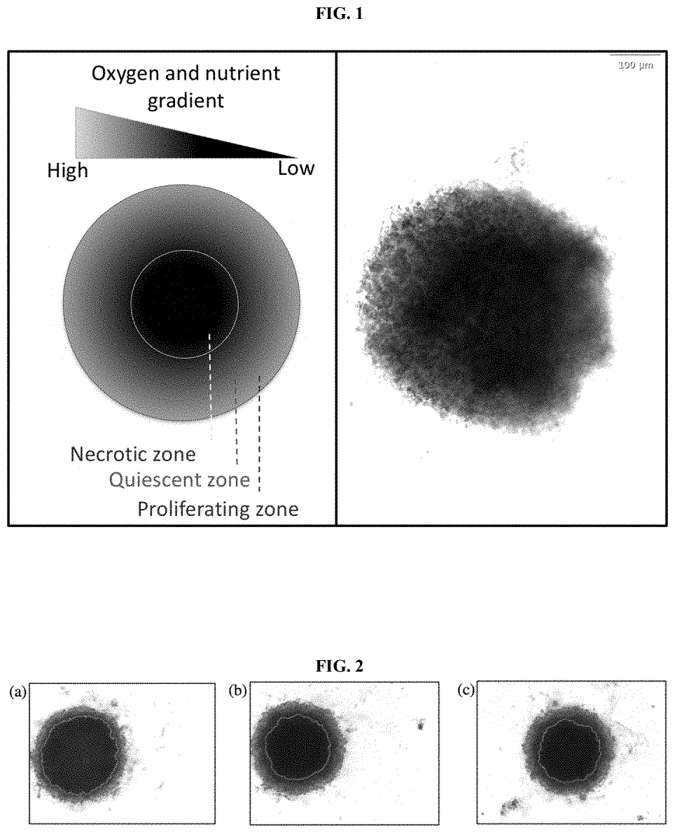

In one example, a well-formed spheroid of at least 500 μm exhibits a concentric structure with distinct proliferating, quiescent and necrotic zones, and a pathophysiological gradient that closely mimic the concentration depreciation of nutrients, oxygen, and metabolites from blood vessels in an in-vivo tumour (FIG. 1).

A major hurdle in using 3D tumour spheroids as a drug-screening model is the lack of automated methods for measuring drug response kinetics in spheroids. Non-image based assays had been developed to determine cell viability or cytotoxicity. These include the use of adenosine triphosphate (ATP) to quantify metabolically active cells, or resazurin reduction to quantify mitochondria metabolic activity, or 4-nitrophenyl phosphate to measure cytosolic acidic phosphatase (APH) levels, or tetrazolium salt to measure Lactate dehydrogenase (LDH) activity, or combinations of these methods. The measurements are mostly based on absorption, luminescence, or fluorescence. Image-based assays typically involve live/dead cell staining and acquiring the images through multiple channels. The need for cell fixation in both non-image and image based assays restrict the assays to end point experiments and limit the ability to monitor pharmacodynamics of the spheroids in response to drug treatments.

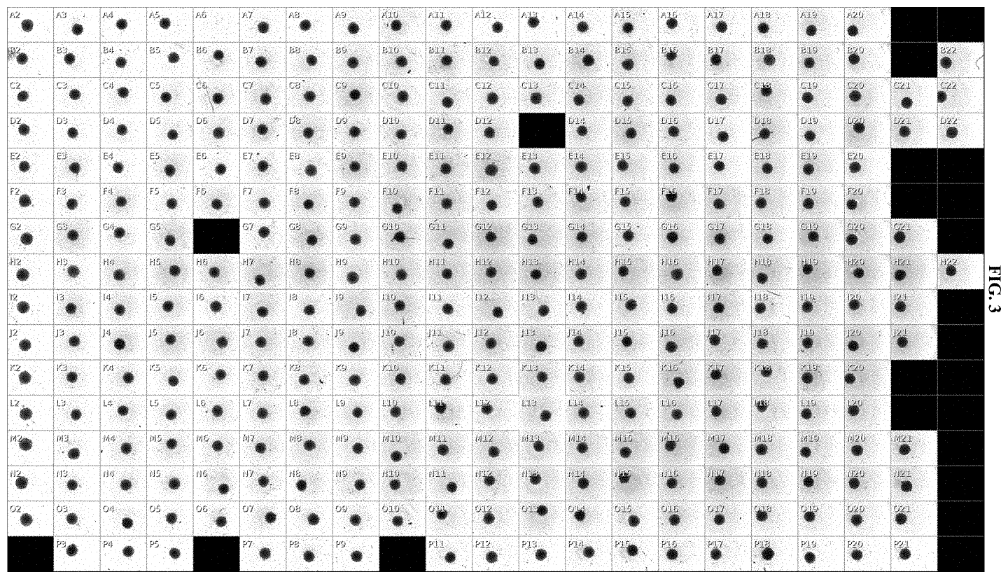

On the other hand, morphological-based methods primarily use the size/volume or shape of the spheroids. However, these parameters do not adequately reflect phenotypic responses. Studies based on tumour spheroids derived from an oral cancer patient (data not shown) revealed that while a tumour spheroid might retain its size and shape upon drug treatment, its internal spatial zone structures have changed in response to the drug activity (FIG. 2).

It is also noted that as the method disclosed herein is based on a predictive model, it is subjected an error rate which is dependent on the accuracy of the computational model and the size and quality of the training samples. The method also assumes that high-resolution images can be obtained of the tumour spheroids. Also the method requires that the spheroids are well formed with distinctive quiescent, necrotic and proliferating zones.

In a similar study involving the same patient, tumour spheroids were generated in a high-throughput 384 well format (FIG. 3). 1,170 spheroids were cultured in four 384 well ultra-low attachment plates and treated with 480 anti-cancer small molecules and kinase inhibitors for 12 types of cancer, some of which are FDA approved.

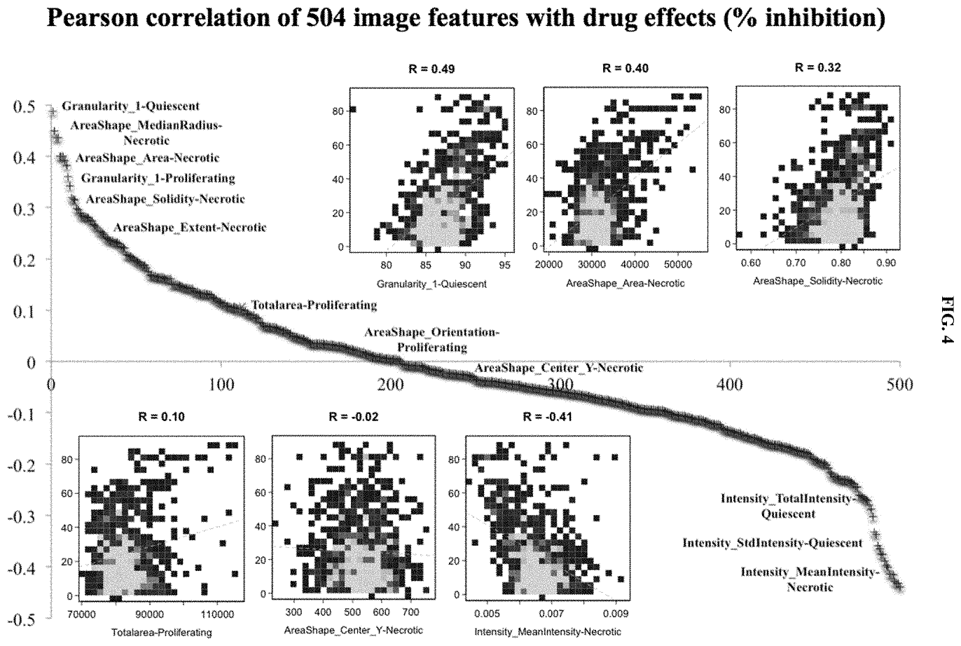

In one example, confocal brightfield images were acquired at 72 hours after drug treatment, and segmented to obtain 504 image measurements from the necrotic, quiescent and proliferating zones of each tumour spheroid, the methods of which are disclosed herein. Correlating each image features separately with the drug response reveals a poor correlation between the size of the spheroids (“TotalArea-Proliferating”, r=0.10) and the efficacy of the drugs on the tumour spheroids (FIG. 4). On the contrary, selected shape, textual and size-associated image features derived from the different zones of the spheroids are better correlated with drug response. These include the area of the necrotic core zone in the spheroid (“AreaShape_Area-Necrotic”, r=0.40), the curvature of the necrotic zone (“AreaShape_Solidity-Necrotic”, r=0.32), and the texture of the quiescent zone (“Granularity_1−Quiescent, r=0.49) are better correlated with drug response. Features that correlate inversely with drug response such as the intensity of the necrotic zone (“Intensity_Meanintensity-Necrotic”, r=−0.41) are also relevant as a proxy for characterizing the response of the spheroids to drugs while logically unrelated features such as the y location of the necrotic zone in the image (“AreaShape_Center_Y-Necrotic”, r=−0.02) show very low correlation with drug response. It is noted that the term “r” as used herein refers to the Pearson's correlation coefficient.

Compared to monolayer cell cultures, tumour spheroids generally show reduced physiochemical response to chemotherapeutic drugs. Many treatments with high efficacy in 2D cell cultures have diminish inhibition activity in 3D tumour spheroids, possibly due to the regulation of tumour microenvironment physiology established by the cell-cell and cell-matrix interactions in a 3D settings and a drug penetration gradient established by the presence of a hypoxic necrotic zone, a quiescent zone that is inherently drug-resistant, and the proliferating zone that is directly exposed to the effect of the drugs.

Thus, in one example, the 3D structure is a spheroid. In another example, the 3D structure is a tumour spheroid.

The 3D tumour spheroids have proven to be a prevailing tool for therapeutic evaluation, both in the negative and positive selection of drugs. As an in-vivo-like models that better recapitulate the primary tumours, tumour spheroid models can be used for high-throughput chemical screening to enable elimination of false positives (of 2D monolayer models), thereby reducing down-stream animal testing. It has also been shown that certain signalling pathways are only activated in a 3D context due to factors such as the presence of cell-matrix interactions. Hence, the tumour spheroid models can be used to identify drugs which have in-vivo efficacy, but whose activity is suppressed in a 2D monolayer models. When tumour spheroids are cultured from the cell-lines derived from a patient or a subject, the tumour spheroids model patient specific factors that can alter the response of drug due to metabolic and micro-environmental differences, and can be a valuable tool for precision oncology studies.

As used herein, the term “agent” includes, but is not limited to, proteins, polypeptides, inorganic molecules, organic molecules (such as small organic molecules), polysaccharides, polynucleotides, and the like. In one example, the agent is, but is not limited to, a substance, a molecule, an element, a compound, an entity or combinations thereof. A list of such agents has been provided in the tables (for example, Tables 1 to 2) as well as in the figures (for example FIG. 22) in the present application.

In another example, an agent can be, but is not limited to, polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs and combinations thereof.

In yet another example, an agent can be one or more synthetic molecules. In yet another example, an agent can be one or more natural molecules. The agents as referred to herein can be obtained from a wide variety of sources, including libraries of synthetic or natural compounds.

In one example, the agent is a polypeptide. In such examples where the agent is a polypeptide, the polypeptide can be about 4 to about 30 amino acids, about 5 to about 20 amino acids, or about 7 to about 15 amino acids in length.

In another example, the agent can be one or more polynucleotides. Examples of such polynucleotides include, but are not limited to, naturally occurring nucleic acids, random nucleic acids, or “biased” random nucleic acids. Further examples of a polynucleotide agent can be, but are not limited to, siRNA, shRNA, a cDNA, gRNA, and combinations thereof.

In another example, an agent can be or include antibodies against molecular targets. Such antibodies can be, but are not limited to, any class of antibody known in the art, for example, IgA, IgD, IgE, IgG, or IgM. As used herein, the term “antibody” refers to an immunoglobulin molecule able to bind to a specific epitope on an antigen. Antibodies can be comprised of a polyclonal mixture, or may be monoclonal in nature. Further, antibodies can be entire immunoglobulins derived from natural sources, or from recombinant sources. The antibodies disclosed herein may exist in a variety of forms, including for example as a whole antibody, or as an antibody fragment, or other immunologically active fragment thereof, such as complementarity determining regions. Similarly, the antibody may exist as an antibody fragment having functional antigen-binding domains, that is, heavy and light chain variable domains. Also, the antibody fragment may exist in a form selected from the group consisting of, but not limited to: Fv, Fab, F(ab)2, scFv (single chain Fv), dAb (single domain antibody), bi-specific antibodies, diabodies and triabodies.

As used herein, the terms “activity” and “response” can be used interchangeably and is used to refer to a biological activity of the agent with regards to the 3D cell structure. The response or activity can include, but is not limited to, inhibitory activity against one or more cells, reducing growth of one or more cells, cytotoxic towards one or more cells, inhibiting proliferation of one or more cell growth, inhibiting differentiation of one or more cell growth and the like.

When testing the activity or response of the 3D cellular structure, the 3D cellular structure and the test agent must come into contact with each other. Also, experimental conditions can require that the 3D structure be exposed or contacted with the test agent for pre-determined amount of time. Thus, in one example, the 3D structure is exposed or subjected to the test agent for a pre-determined or determined amount of time.

In some examples, the features as described herein includes, but is not limited to, features as listed below. As would be understood by the person skilled in the art, feature selection in the methods as described herein may be performed by methods that are known in the art. In some examples, in the methods as described herein, the feature is selected using methods such as, but is not limited to, correlation feature selection (for example CFS with cut-off 0.5), entropy-based selection, mutual information, best first, genetic algorithm, greedy stepwise selection for subset selection, and the like. It would also be within the skill of the person in the art to determine the cut-off of acceptable threshold for each feature to be selected. For example, it would be readily understood that the cut-off may differ depending on the dataset, and each dataset will have slightly different optimized parameters.

This is a novel image segmentation and analysis method, as disclosed herein, comprehensively exploits the morphology of different zones (using different image parameters such as textual, intensity, etc.) of tumour spheroids to construct a quantitative model of the spheroid's sensitivity to drugs. Morphological changes can be measured from bright-field/digital phase contrast images of the tumour spheroids. Importantly, the method disclosed herein enables a label-free method to continuous monitor the response kinetics of a single tumour spheroid, and to comprehensive profile of the pharmacokinetics of a drug in 3D tumour models.

Using the approach disclosed herein, the feasibility of (1) segmenting the brightfield/digital-phase contrast images to extract multiple spheroid zone-specific image features, (2) training of classifier with machine learning to identify/determine drug response and (3) comparing with standard cytotoxicity measurements (using 3-D Cell titre GLO as a validation of proof-of-principle using multiple standard of care drugs that are currently in the clinic) has been clearly shown.

Applied to tumour spheroids derived from a patient, the method and system disclosed herein allow for quantitative profiling of the specific response kinetics of the patient's tumour spheroids to a wide spectrum of drugs. This enables a comprehensive assessment of different therapeutic options for individual patients and provides enhanced information for therapeutic selection, thus facilitating personalized oncology and precision therapy.

In summary, the pitfalls of using spheroid size or volume in predicting drug response have been shown. Comparatively, zone-specific image descriptors are more predictive of the pharmacological response of the tumour spheroids but with limited predictive value (r<0.5). More complex image descriptors will be required to (1) attain more accurate quantification of the drug response at each time-point, and to (2) profile the kinetics of the drug response over time. The method and system disclosed herein exploits morphological changes in the zonal structures of tumour spheroids, and utilizes machine-learning methods to generate multivariate models of image features with improved predictability of the drug response. Given that the images are acquired from bright-field images, a label-free, non-invasive system can be established for continuous monitoring of the response kinetics of the 3D spheroids in a high-throughput set-up.

As used in this application, the singular form “a,” “an,” and “the” include plural references unless the context clearly dictates otherwise. For example, the term “a genetic marker” includes a plurality of genetic markers, including mixtures and combinations thereof.

The word “substantially” does not exclude “completely” e.g. a composition which is “substantially free” from Y may be completely free from Y. Where necessary, the word “substantially” may be omitted from the definition of the invention.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention illustratively described herein may suitably be practiced in the absence of any element or elements, limitation or limitations, not specifically disclosed herein. Thus, unless specified otherwise, the terms “comprising,” “including,” and “containing,” and grammatical variants thereof, are intended to represent “open” or “inclusive” language such that they include recited elements but also permit inclusion of additional, un-recited elements. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

The word “substantially” does not exclude “completely” e.g. a composition which is “substantially free” from Y may be completely free from Y. Where necessary, the word “substantially” may be omitted from the definition of the invention.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention illustratively described herein may suitably be practiced in the absence of any element or elements, limitation or limitations, not specifically disclosed herein. Thus, unless specified otherwise, the terms “comprising,” “including,” and “containing,” and grammatical variants thereof, are intended to represent “open” or “inclusive” language such that they include recited elements but also permit inclusion of additional, un-recited elements. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

### Tables

TABLE 1
Anticancer Drug Library from Selleck

| Compound | Row | Column | Target | Pathway |
|---|---|---|---|---|
| ABT-737 | A | 3 | Bcl-2 | Apoptosis |
| Andarine (GTX-007) | A | 4 | Androgen Receptor | |
| Linifanib (ABT-869) | A | 5 | PDGFR, VEGFR | Protein Tyrosine |
| | | | | Kinase |
| 17-AAG (Tanespimycin) | A | 6 | HSP | |
| ABT-888 (Veliparib) | A | 7 | PARP | DNA Damage |
| 17-DMAG HCl (Alvespimycin) | A | 8 | HSP | |
| Axitinib | A | 9 | VEGFR, PDGFR, c-Kit | Protein Tyrosine |
| | | | | Kinase |
| SNS-032 (BMS-387032) | A | 10 | CDK | |
| Saracatinib (AZD0530) | A | 11 | Src, Bcr-Abl | Angiogenesis |
| Cyclopamine | A | 12 | Hedgehog | |
| AZD6244 (Selumetinib) | A | 13 | MEK | MAPK |
| Barasertib (AZD1152-HQPA) | A | 14 | Aurora Kinase | |
| BEZ235 (NVP-BEZ235) | A | 15 | ATM/ATR, mTOR, | |
| | | | PI3K | |
| Docetaxel (Taxotere) | A | 16 | Microtubule | |
| | | | Associated | |
| BIBF1120 (Vargatef) | A | 17 | VEGFR, PDGFR, | Protein Tyrosine |
| | | | FGFR | Kinase |
| Gemcitabine HCl (Gemzar) | A | 18 | Autophagy | |
| Afatinib (BIBW2992) | A | 19 | EGFR | Protein Tyrosine |
| | | | | Kinase |
| Paclitaxel (Taxol) | A | 20 | Microtubule | |
| | | | Associated | |
| Bortezomib (Velcade) | A | 21 | Proteasome | Proteases |
| Roscovitine (Seliciclib, CYC202) | A | 22 | CDK | |
| Rigosertib (ON-01910) | B | 3 | PLK | Cell Cycle |
| TAME | B | 4 | APC | Cell Cycle |
| Epothilone B (EPO906) | B | 5 | Microtubule | Cytoskeletal |
| | | | Associated | Signaling |
| CAL-101 (GS-1101) | B | 6 | PI3K | PI3K/Akt/mTOR |
| Dorzolamide HCl | B | 7 | Carbonic Anhydrase | |
| LY2157299 | B | 8 | TGF-beta/Smad | TGF-beta/Smad |
| Ruxolitinib (INCB018424) | B | 9 | JAK | JAK/STAT |
| Telatinib (BAY 57-9352) | B | 10 | VEGFR, PDGFR, c-Kit | Protein Tyrosine |
| | | | | Kinase |
| Isotretinoin | B | 11 | Hydroxylase | Metabolism |
| BI6727 (Volasertib) | B | 12 | PLK | Cell Cycle |
| Pelitinib (EKB-569) | B | 13 | EGFR | Protein Tyrosine |
| | | | | Kinase |
| Palomid 529 | B | 14 | mTOR, PI3K | |
| Zileuton | B | 15 | | |
| WP1130 | B | 16 | DUB, Bcr-Abl | Angiogenesis |
| Ispinesib (SB-715992) | B | 17 | Kinesin | Cytoskeletal |
| | | | | Signaling |
| AR-42 (HDAC-42) | B | 18 | HD AC | |
| Tipifarnib (Zarnestra) | B | 19 | Farnesyltransferase, | Metabolism |
| | | | Ras | |
| CP-466722 | B | 20 | ATM/ATR | |
| Zibotentan (ZD4054) | B | 21 | ETA Receptor | GPCR&G |
| | | | | Protein |
| BKM120 (NVP-BKM120) | B | 22 | PI3K | PI3K/Akt/mTOR |
| Cediranib (AZD2171) | C | 3 | VEGFR, Flt | Protein Tyrosine |
| | | | | Kinase |
| Capecitabine (Xeloda) | C | 4 | DNA/RNA Synthesis | |
| Dovitinib (TKI-258) | C | 5 | c-Kit, FGFR, Flt, | Angiogenesis |
| | | | VEGFR, PDGFR | |
| Ganetespib (STA-9090) | C | 6 | HSP | |
| CI-1040 (PD184352) | C | 7 | MEK | MAPK |
| E7080 (Lenvatinib) | C | 8 | VEGFR | Protein Tyrosine |
| | | | | Kinase |
| Dasatinib (BMS-354825) | C | 9 | Src, Bcr-Abl, c-Kit | Angiogenesis |
| ABT-751 | C | 10 | Microtubule | Cytoskeletal |
| | | | Associated | Signaling |
| Deforolimus (Ridaforolimus) | C | 11 | mTOR | |
| Cisplatin | C | 12 | DNA/RNA Synthesis | |
| Erlotinib HCl | C | 13 | EGFR | Protein Tyrosine |
| | | | | Kinase |
| Valproic acid sodium salt | C | 14 | GABA Receptor, | Neuronal |
| (Sodium valproate) | | | HD AC | Signaling |
| Gefitinib (Iressa) | C | 15 | EGFR | Protein Tyrosine |
| | | | | Kinase |
| CYC116 | C | 16 | Aurora Kinase, | Cell Cycle |
| | | | VEGFR | |
| Imatinib Mesylate | C | 17 | PDGFR, c-Kit, Bcr-Abl | Protein Tyrosine |
| | | | | Kinase |
| JNJ 26854165 (Serdemetan) | C | 18 | p53 | Apoptosis |
| Lapatinib Ditosylate (Tykerb) | C | 19 | EGFR, HER2 | Protein Tyrosine |
| | | | | Kinase |
| WZ4002 | C | 20 | EGFR | Protein Tyrosine |
| | | | | Kinase |
| Lenalidomide (Revlimid) | C | 21 | TNF-alpha | Apoptosis |
| Ostarine (MK-2866) | C | 22 | Androgen Receptor | Endocrinology & |
| | | | | Hormones |
| SB 525334 | D | 3 | TGF-beta/Smad | TGF-beta/Smad |
| (−)-Epigallocatechin gallate | D | 4 | | |
| AEE788 (NVP-AEE788) | D | 5 | EGFR, Flt, VEGFR, | Protein Tyrosine |
| | | | HER2 | Kinase |
| Cyclosporin A (Cyclosporine A) | D | 6 | | |
| PHA-793887 | D | 7 | CDK | Cell Cycle |
| Gossypol | D | 8 | Dehydrogenase | |
| PIK-93 | D | 9 | PI3K, VEGFR | PI3K/Akt/mTOR |
| Phloretin (Dihydronaringenin) | D | 10 | | |
| Ponatinib (AP24534) | D | 11 | Bcr-Abl, VEGFR, | Angiogenesis |
| | | | FGFR, PDGFR, Flt | |
| Salinomycin (Procoxacin) | D | 12 | | |
| Fludarabine (Fludara) | D | 13 | STAT, DNA/RNA | JAK/STAT |
| | | | Synthesis | |
| Quercetin (Sophoretin) | D | 14 | PI3K, PKC, Src, Sirtuin | Epigenetics |
| LY2228820 | D | 15 | p38 MAPK | MAPK |
| Coenzyme Q10 (CoQ10) | D | 16 | | |
| Mycophenolate mofetil (CellCept) | D | 17 | | Metabolism |
| Chrysophanic acid | D | 18 | EGFR, mTOR | Protein Tyrosine |
| (Chrysophanol) | | | | Kinase |
| SB939 (Pracinostat) | D | 19 | HD AC | Cytoskeletal |
| | | | | Signaling |
| Imatinib (Gleevec) | D | 20 | PDGFR, c-Kit, v-Abl | Protein Tyrosine |
| | | | | Kinase |
| Tosedostat (CHR2797) | D | 21 | Aminopeptidase | |
| Itraconazole (Sporanox) | D | 22 | | |
| Nilotinib (AMN-107) | E | 3 | Bcr-Abl | Angiogenesis |
| Regorafenib (BAY 73-4506) | E | 4 | c-Kit, Raf, VEGFR | Protein Tyrosine |
| | | | | Kinase |
| PD0325901 | E | 5 | MEK | DNA Damage |
| XAV-939 | E | 6 | Wnt/beta-catenin | Stem Cells & |
| | | | | Wnt |
| PI-103 | E | 7 | DNA-PK, PI3K, mTOR | Neuronal |
| | | | | Signaling |
| ENMD-2076 | E | 8 | Flt, Aurora Kinase, | Angiogenesis |
| | | | VEGFR | |
| Rapamycin (Sirolimus) | E | 9 | mTOR | DNA Damage |
| BIBR 1532 | E | 10 | Telomerase | DNA Damage |
| Sorafenib (Nexavar) | E | 11 | VEGFR, PDGFR, Raf | Neuronal |
| | | | | Signaling |
| PIK-90 | E | 12 | PI3K | |
| STF-62247 | E | 13 | | |
| Anastrozole | E | 14 | Aromatase | Endocrinology & |
| | | | | Hormones |
| Sunitinib Malate (Sutent) | E | 15 | VEGFR, PDGFR, c- | Microbiology |
| | | | Kit, Flt | |
| Aprepitant (MK-0869) | E | 16 | Substance P | |
| Tandutinib (MLN518) | E | 17 | Flt | |
| Bicalutamide (Casodex) | E | 18 | Androgen Receptor, | Endocrinology & |
| | | | P450 | Hormones |
| Temsirolimus (Torisel) | E | 19 | mTOR | Neuronal |
| | | | | Signaling |
| Fulvestrant (Faslodex) | E | 20 | Estrogen/progestogen | Endocrinology & |
| | | | Receptor | Hormones |
| Trichostatin A (TSA) | E | 21 | HD AC | |
| Raltitrexed (Tomudex) | E | 22 | DNA/RNA Synthesis | DNA Damage |
| AT7519 | F | 3 | CDK | Cell Cycle |
| Mycophenolic (Mycophenolate) | F | 4 | | |
| MK-1775 | F | 5 | Wee1 | Cell Cycle |
| Rosiqlitazone (Avandia) | F | 6 | PPAR | DNA Damage |
| Quizartinib (AC220) | F | 7 | Flt | Angiogenesis |
| Medroxyprogesterone acetate | F | 8 | Estrogen/progestogen | Endocrinology & |
| | | | Receptor | Hormones |
| AZD7762 | F | 9 | Chk | Cell Cycle |
| Pioqlitazone (Actos) | F | 10 | | |
| R406 (free base) | F | 11 | Syk | Angiogenesis |
| Mifepristone (Mifeprex) | F | 12 | Estrogen/progestogen | Endocrinology & |
| | | | Receptor | Hormones |
| DMXAA (ASA404) | F | 13 | VDA | Angiogenesis |
| Lonidamine | F | 14 | | |
| EX 527 | F | 15 | Sirtuin | Epigenetics |
| TAK-733 | F | 16 | MEK | MAPK |
| Febuxostat (Uloric) | F | 17 | | |
| LDN193189 | F | 18 | TGF-beta/Smad | |
| Dapagliflozin | F | 19 | SGLT | GPCR&G |
| | | | | Protein |
| LY2603618 (IC-83) | F | 20 | Chk | Cell Cycle |
| AZD8055 | F | 21 | mTOR | PI3K/Akt/mTOR |
| GW3965 HCl | F | 22 | Liver X Receptor | |
| Vorinostat (SAHA) | G | 3 | HD AC | Endocrinology & |
| | | | | Hormones |
| CUDC-101 | G | 4 | HDAC, EGFR, HER2 | Epigenetics |
| VX-680 (MK-0457, Tozasertib) | G | 5 | Aurora Kinase | Endocrinology & |
| | | | | Hormones |
| Exemestane | G | 6 | Aromatase | Endocrinology & |
| | | | | Hormones |
| Y-27632 2HCl | G | 7 | ROCK | |
| Irinotecan | G | 8 | Topoisomerase | DNA Damage |
| Elesclomol | G | 9 | HSP | Angiogenesis |
| Cladribine | G | 10 | DNA/RNA Synthesis | DNA Damage |
| Entinostat (MS-275, SNDX-275) | G | 11 | HDAC | Transmembrane |
| | | | | Transporters |
| Decitabine | G | 12 | DNA/RNA Synthesis | Epigenetics |
| Enzastaurin (LY317615) | G | 13 | PKC | Neuronal |
| | | | | Signaling |
| Dimesna | G | 14 | | |
| BMS-599626 (AC480) | G | 15 | HER2 | Neuronal |
| | | | | Signaling |
| PIK-75 | G | 16 | PI3K, DNA-PK | PI3K/Akt/mTOR |
| Obatoclax mesylate (GX15-070) | G | 17 | Bcl-2 | Neuronal |
| | | | | Signaling |
| Tivozanib (AV-951) | G | 18 | VEGFR, c-Kit, PDGFR | Protein Tyrosine |
| | | | | Kinase |
| Olaparib (AZD2281) | G | 19 | PARP | Protein Tyrosine |
| | | | | Kinase |
| Doxorubicin (Adriamycin) | G | 20 | Topoisomerase | DNA Damage |
| Nutlin-3 | G | 21 | Mdm2 | Neuronal |
| | | | | Signaling |
| Adrucil (Fluorouracil) | G | 22 | DNA/RNA Synthesis | DNA Damage |
| Pomalidomide | H | 3 | TNF-alpha, COX | Apoptosis |
| NU7441 (KU-57788) | H | 4 | DNA-PK, PI3K | DNA Damage |
| KU-60019 | H | 5 | ATM | DNA Damage |
| GSK2126458 | H | 6 | PI3K, mTOR | PI3K/Akt/mTOR |
| BIRB 796 (Doramapimod) | H | 7 | p38 MAPK | MAPK |
| MK-0752 | H | 8 | Gamma-secretase | Proteases |
| Tie2 kinase inhibitor | H | 9 | Tie-2 | Protein Tyrosine |
| | | | | Kinase |
| PF-3845 | H | 10 | FAAH | Metabolism |
| Ubenimex (Bestatin) | H | 11 | | |
| GSK1120212 (Trametinib) | H | 12 | MEK | MAPK |
| Prednisone (Adasone) | H | 13 | | |
| Flavopiridol (Alvocidib) HCl | H | 14 | CDK | Cell Cycle |
| Triamcinolone Acetonide | H | 15 | | |
| PCI-32765 (Ibrutinib) | H | 16 | Src | Angiogenesis |
| Cytarabine | H | 17 | DNA/RNA Synthesis | |
| NVP-BSK805 2HCl | H | 18 | JAK | JAK/STAT |
| Tretinoin (Aberela) | H | 19 | | |
| WAY-362450 | H | 20 | FXR | |
| Ezetimibe (Zetia) | H | 21 | | |
| A-769662 | H | 22 | AMPK | PI3K/Akt/mTOR |
| GDC-0941 | I | 3 | PI3K | Metabolism |
| Imiquimod | I | 4 | | |
| SB 431542 | I | 5 | TGF-beta/Smad | |
| Bendamustine HCL | I | 6 | | |
| Crizotinib (PF-02341066) | I | 7 | c-Met, ALK | |
| Nelarabine (Arranon) | I | 8 | DNA/RNA Synthesis | DNA Damage |
| AUY922 (NVP-AUY922) | I | 9 | HSP | |
| Bleomycin sulfate | I | 10 | DNA/RNA Synthesis | DNA Damage |
| PHA-665752 | I | 11 | c-Met | |
| Carboplatin | I | 12 | DNA/RNA Synthesis | |
| ZSTK474 | I | 13 | PI3K | Neuronal |
| | | | | Signaling |
| Clafen (Cyclophosphamide) | I | 14 | DNA/RNA Synthesis | |
| SB 216763 | I | 15 | GSK-3 | |
| Clofarabine | I | 16 | DNA/RNA Synthesis | DNA Damage |
| SB 203580 | I | 17 | p38 MAPK | Transmembrane |
| | | | | Transporters |
| YM201636 | I | 18 | PI3K | PI3K/Akt/mTOR |
| MK-2206 2HCl | I | 19 | Akt | |
| OSI-930 | I | 20 | c-Kit, VEGFR | Protein Tyrosine |
| | | | | Kinase |
| PD153035 HCl | I | 21 | EGFR | |
| Dacarbazine (DTIC-Dome) | I | 22 | DNA/RNA Synthesis | DNA Damage |
| Aminoglutethimide (Cytadren) | J | 3 | Aromatase | Endocrinology & |
| | | | | Hormones |
| KX2-391 | J | 4 | Src | Angiogenesis |
| Disulfiram (Antabuse) | J | 5 | | |
| LY2109761 | J | 6 | TGF-beta/Smad | |
| Betapar (Meprednisone) | J | 7 | | |
| YO-01027 | J | 8 | Gamma-secretase | Proteases |
| Busulfan (Myleran, Busulfex) | J | 9 | | |
| Geldanamycin | J | 10 | HSP | Cytoskeletal |
| | | | | Signaling |
| Hydrocortisone (Cortisol) | J | 11 | | |
| AMG 900 | J | 12 | Aurora Kinase | Cell Cycle |
| Estradiol | J | 13 | | |
| PF-03814735 | J | 14 | Aurora Kinase | |
| Gemcitabine (Gemzar) | J | 15 | | DNA Damage |
| PH-797804 | J | 16 | p38 MAPK | MAPK |
| Azathioprine (Azasan, Imuran) | J | 17 | | |
| Dacomitinib (PF299804, PF- | J | 18 | EGFR | Protein Tyrosine |
| 00299804) | | | | Kinase |
| Mesna (Uromitexan, Mesnex) | J | 19 | | |
| Crenolanib (CP-868596) | J | 20 | PDGFR | Protein Tyrosine |
| | | | | Kinase |
| Toremifene Citrate (Fareston, | J | 21 | | Endocrinology & |
| Acapodene) | | | | Hormones |
| AZ 3146 | J | 22 | Kinesin | Cytoskeletal |
| | | | | Signaling |
| Vismodegib (GDC-0449) | K | 3 | Hedgehog, P-gp | Neuronal |
| | | | | Signaling |
| Oxaliplatin (Eloxatin) | K | 4 | DNA/RNA Synthesis | DNA Damage |
| Brivanib (BMS-540215) | K | 5 | VEGFR, FGFR | GPCR&G |
| | | | | Protein |
| Etoposide (VP-16) | K | 6 | Topoisomerase | DNA Damage |
| Belinostat (PXD101) | K | 7 | HD AC | |
| Ku-0063794 | K | 8 | mTOR | PI3K/Akt/mTOR |
| Iniparib (BSI-201) | K | 9 | PARP | |
| Evista (Raloxifene HCl) | K | 10 | Estrogen/progestogen | Endocrinology & |
| | | | Receptor | Hormones |
| PCI-24781 | K | 11 | HD AC | |
| Idarubicin HCl | K | 12 | Topoisomerase | DNA Damage |
| Linsitinib (OSI-906) | K | 13 | IGF-1R | |
| Fludarabine Phosphate (Fludara) | K | 14 | DNA/RNA Synthesis | DNA Damage |
| KU-55933 | K | 15 | ATM | |
| Topotecan HCl | K | 16 | Topoisomerase | DNA Damage |
| GSK1904529A | K | 17 | IGF-1R | |
| 2-Methoxyestradiol | K | 18 | HIF | Angiogenesis |
| PF-04217903 | K | 19 | c-Met | |
| Letrozole | K | 20 | Aromatase | Endocrinology & |
| | | | | Hormones |
| JNJ-26481585 | K | 21 | HD AC | |
| Leucovorin Calcium | K | 22 | | |
| Teniposide (Vumon) | L | 3 | | |
| PAC-1 | L | 4 | Caspase | Apoptosis |
| Simvastatin (Zocor) | L | 5 | | |
| AZ628 | L | 6 | Raf | MAPK |
| Ranolazine (Ranexa) | L | 7 | | |
| AT-406 | L | 8 | E3 Ligase | |
| Lomustine (CeeNU) | L | 9 | | |
| Canagliflozin | L | 10 | SGLT | GPCR&G |
| | | | | Protein |
| D-glutamine | L | 11 | | |
| 3-Methyladenine | L | 12 | PI3K | PI3K/Akt/mTOR |
| Hydroxyurea (Cytodrox) | L | 13 | | |
| Dalcetrapib (JTT-705) | L | 14 | CETP | Metabolism |
| Flutamide (Eulexin) | L | 15 | P450 | Endocrinology & |
| | | | | Hormones |
| Nocodazole | L | 16 | Microtubule | Cytoskeletal |
| | | | Associated | Signaling |
| Fluvastatin sodium (Lescol) | L | 17 | HMG-CoA Reductase | Metabolism |
| GW4064 | L | 18 | FXR | |
| Tamoxifen Citrate (Nolvadex) | L | 19 | Estrogen/progestogen | Endocrinology & |
| | | | Receptor | Hormones |
| Tofacitinib (CP-690550, | L | 20 | JAK | JAK/STAT |
| Tasocitinib) | | | | |
| Procarbazine HCl (Matulane) | L | 21 | DNA/RNA Synthesis | |
| Sotrastaurin (AEB071) | L | 22 | PKC | TGF-beta/Smad |
| Rucaparib (AG-014699 , PF- | M | 3 | PARP | |
| 01367338) | | | | |
| Vincristine | M | 4 | Autophagy, Microtubule | Cytoskeletal |
| | | | Associated | Signaling |
| Vatalanib 2HCl (PTK787) | M | 5 | VEGFR, c-Kit, Flt | |
| Amuvatinib (MP-470) | M | 6 | c-Met, c-Kit, PDGFR, | Protein Tyrosine |
| | | | Flt, c-RET | Kinase |
| GDC-0879 | M | 7 | Raf | |
| Vinblastine | M | 8 | AChR | |
| LY294002 | M | 9 | PI3K | |
| JNJ-7706621 | M | 10 | CDK, Aurora Kinase | Cell Cycle |
| Danusertib (PHA-739358) | M | 11 | Aurora Kinase, FGFR, | |
| | | | Bcr-Abl, c-RET, Src | |
| MDV3100 (Enzalutamide) | M | 12 | Androgen Receptor, | Endocrinology & |
| | | | P450 | Hormones |
| TAE684 (NVP-TAE684) | M | 13 | ALK | |
| Celecoxib | M | 14 | COX | Neuronal |
| | | | | Signaling |
| BI 2536 | M | 15 | PLK | |
| PD173074 | M | 16 | FGFR, VEGFR | Angiogenesis |
| SGX-523 | M | 17 | c-Met | |
| WYE-354 | M | 18 | mTOR | PI3K/Akt/mTOR |
| GSK690693 | M | 19 | Akt | |
| Vemurafenib (PLX4032) | M | 20 | Raf | MAPK |
| JNJ-38877605 | M | 21 | c-Met | |
| IC-87114 | M | 22 | PI3K | |
| Maraviroc | N | 3 | CCR5 | Microbiology |
| Sirtinol | N | 4 | Sirtuin | Epigenetics |
| PF 573228 | N | 5 | FAK | Angiogenesis |
| CEP33779 | N | 6 | JAK | JAK/STAT |
| Cyclophosphamide monohydrate | N | 7 | | |
| INK 128 (MLN0128) | N | 8 | mTOR | PI3K/Akt/mTOR |
| Bexarotene | N | 9 | | |
| Torin 2 | N | 10 | mTOR | PI3K/Akt/mTOR |
| Vinpocetine (Cavinton) | N | 11 | Sodium Channel | |
| RG108 | N | 12 | Transferases | Epigenetics |
| Lapatinib | N | 13 | EGFR, HER2 | Protein Tyrosine |
| | | | | Kinase |
| TPCA-1 | N | 14 | IKK | NF-?B |
| Neratinib (HKI-272) | N | 15 | HER2, EGFR | |
| Desmethyl Erlotinib (CP-473420) | N | 16 | EGFR | |
| LDE225 (NVP-LDE225, | N | 17 | Smoothened | Stem Cells & |
| Erismodegib) | | | | Wnt |
| Torin 1 | N | 18 | Autophagy, mTOR | |
| AG14361 | N | 19 | PARP | DNA Damage |
| PF-562271 | N | 20 | FAK | Angiogenesis |
| MLN2238 | N | 21 | Proteasome | Proteases |
| S-Ruxolitinib | N | 22 | JAK | JAK/STAT |
| Triciribine (Triciribine phosphate) | O | 3 | Akt | |
| Altretamine (Hexalen) | O | 4 | | |
| XL147 | O | 5 | PI3K | |
| Carmofur | O | 6 | Antimetabolites | DNA Damage |
| Everolimus (RAD001) | O | 7 | mTOR | |
| Epothilone A | O | 8 | Microtubule | Cytoskeletal |
| | | | Associated | Signaling |
| TW-37 | O | 9 | Bcl-2 | |
| Floxuridine (Fludara) | O | 10 | DNA/RNA Synthesis | DNA Damage |
| Mocetinostat (MGCD0103) | O | 11 | HD AC | |
| Ftorafur | O | 12 | DNA/RNA Synthesis | DNA Damage |
| Abiraterone (CB-7598) | O | 13 | P450 (e.g. CYP17) | |
| Ifosfamide | O | 14 | DNA/RNA Synthesis | DNA Damage |
| SRT1720 | O | 15 | Sirtuin | |
| Megestrol Acetate | O | 16 | Androgen Receptor | Endocrinology & |
| | | | | Hormones |
| YM155 | O | 17 | Survivin | |
| Mercaptopurine | O | 18 | DNA/RNA Synthesis | DNA Damage |
| MLN8237 (Alisertib) | O | 19 | Aurora Kinase | |
| Pamidronate Disodium | O | 20 | | |
| AT9283 | O | 21 | Bcr-Abl, JAK, Aurora | |
| | | | Kinase | |
| Streptozotocin (Zanosar) | O | 22 | | |
| SB 743921 | P | 3 | Kinesin | |
| CHIR-99021 (CT99021) HCl | P | 4 | PI3K/Akt/mTOR | PI3K/Akt/mTOR |
| GSK461364 | P | 5 | PLK | Cell Cycle |
| Pazopanib | P | 6 | VEGFR | Protein Tyrosine |
| | | | | Kinase |
| SGI-1776 free base | P | 7 | Pim | JAK/STAT |
| Daunorubicin HCl (Daunomycin | P | 8 | Telomerase | DNA Damage |
| HCl) | | | | |
| BMS 794833 | P | 9 | c-Met, VEGFR | Protein Tyrosine |
| | | | | Kinase |
| Dexamethasone acetate | P | 10 | | |
| OSI-420 | P | 11 | EGFR | Protein Tyrosine |
| | | | | Kinase |
| Anagrelide HCl | P | 12 | PDE | Metabolism |
| R935788 (Fostamatinib disodium, | P | 13 | Syk | |
| R788 disodium) | | | | |
| Triptolide | P | 14 | | |
| Formestane | P | 15 | Aromatase | Endocrinology & |
| | | | | Hormones |
| Fingolimod (FTY720) | P | 16 | S1P Receptor, Bcr- | GPCR&G |
| | | | Abl, PKC | Protein |
| DAPT (GSI-IX) | P | 17 | Gamma-secretase, | Proteases |
| | | | Beta Amyloid | |
| Irinotecan HCl Trihydrate | P | 19 | Topoisomerase | DNA Damage |
| (Campto) | | | | |
| Cyt387 | P | 21 | JAK | JAK/STAT |

TABLE 2
Kinase Inhibitor Drug Library from Selleck

| Compound | Row | Column | Target | Pathway |
|---|---|---|---|---|
| Linifanib (ABT- | A | 3 | PDGFR, VEGFR | Protein Tyrosine |
| 869) | | | | Kinase |
| E7080 | A | 4 | VEGFR | Protein Tyrosine |
| (Lenvatinib) | | | | Kinase |
| Cediranib | A | 5 | VEGFR, Flt | Protein Tyrosine |
| (AZD2171) | | | | Kinase |
| WZ8040 | A | 6 | EGFR | Protein Tyrosine |
| | | | | Kinase |
| Imatinib | A | 7 | PDGFR, c-Kit, | Protein Tyrosine |
| Mesylate | | | Bcr-Abl | Kinase |
| AG-1024 | A | 8 | IGF-1R | Protein Tyrosine |
| | | | | Kinase |
| Rapamycin | A | 9 | mTOR | DNA Damage |
| (Sirolimus) | | | | |
| BX-912 | A | 10 | PDK-1 | PI3K/Akt/mTOR |
| Enzastaurin | A | 11 | PKC | Neuronal |
| (LY317615) | | | | Signaling |
| Pelitinib (EKB- | A | 12 | EGFR | Protein Tyrosine |
| 569) | | | | Kinase |
| SB 216763 | A | 13 | GSK-3 | |
| TSU-68 | A | 14 | VEGFR, | Protein Tyrosine |
| | | | PDGFR, FGFR | Kinase |
| Linsitinib (OSI- | A | 15 | IGF-1R | |
| 906) | | | | |
| LY2228820 | A | 16 | p38 MAPK | MAPK |
| GDC-0879 | A | 17 | Rat | |
| AZD7762 | A | 18 | Chk | Cell Cycle |
| GSK690693 | A | 19 | Akt | |
| PD318088 | A | 20 | MEK | MAPK |
| AT9283 | A | 21 | Bcr-Abl, JAK, | |
| | | | Aurora Kinase | |
| Neratinib (HKI- | A | 22 | HER2, EGFR | |
| 272) | | | | |
| R406 | B | 3 | Syk, Flt | Angiogenesis |
| Wortmannin | B | 4 | PI3K | PI3K/Akt/mTOR |
| Mubritinib (TAK | B | 5 | HER2 | Protein Tyrosine |
| 165) | | | | Kinase |
| AZD2014 | B | 6 | mTOR | PI3K/Akt/mTOR |
| BI6727 | B | 7 | PLK | Cell Cycle |
| (Volasertib) | | | | |
| Dabrafenib | B | 8 | Raf | MAPK |
| (GSK2118436) | | | | |
| Phenformin | B | 9 | AMPK | PI3K/Akt/mTOR |
| hydrochloride | | | | |
| TPCA-1 | B | 10 | IKK | NF-?B |
| PF-05212384 | B | 11 | mTOR, PI3K | |
| (PKI-587) | | | | |
| WHI-P154 | B | 12 | JAK, EGFR | JAK/STAT |
| A-674563 | B | 13 | Akt, CDK, PKA | PI3K/Akt/mTOR |
| ABT-263 | B | 14 | Bcl-2 | Apoptosis |
| (Navitoclax) | | | | |
| CHIR-124 | B | 15 | Chk | Cell Cycle |
| Pemetrexed | B | 16 | DHFR | |
| (Alimta) | | | | |
| TAK-901 | B | 17 | Aurora Kinase | Cell Cycle |
| Dexamethasone | B | 18 | IL Receptor | |
| Crenolanib (CP- | B | 19 | PDGFR | Protein Tyrosine |
| 868596) | | | | Kinase |
| SB590885 | B | 20 | Raf | MAPK |
| CHIR-98014 | B | 21 | GSK-3 | PI3K/Akt/mTOR |
| Axitinib | C | 3 | VEGFR, | Protein Tyrosine |
| | | | PDGFR, c-Kit | Kinase |
| CP-724714 | C | 4 | EGFR, HER2 | Protein Tyrosine |
| | | | | Kinase |
| Dovitinib (TKI- | C | 5 | c-Kit, FGFR, Flt, | Angiogenesis |
| 258) | | | VEGFR, PDGFR | |
| ENMD-2076 | C | 6 | Flt, Aurora | Angiogenesis |
| | | | Kinase, VEGFR | |
| Lapatinib | C | 7 | EGFR, HER2 | Protein Tyrosine |
| Ditosylate | | | | Kinase |
| (Tykerb) | | | | |
| Amuvatinib (MP- | C | 8 | c-Met, c-Kit, | Protein Tyrosine |
| 470) | | | PDGFR, Flt, c- | Kinase |
| | | | RET | |
| Sorafenib | C | 9 | VEGFR, | Neuronal |
| (Nexavar) | | | PDGFR, Raf | Signaling |
| AMG-208 | C | 10 | c-Met | |
| BMS-599626 | C | 11 | HER2 | Neuronal |
| (AC480) | | | | Signaling |
| AS-605240 | C | 12 | PI3K | |
| SB 203580 | C | 13 | p38 MAPK | Transmembrane |
| | | | | Transporters |
| AS703026 | C | 14 | MEK | MAPK |
| KU-55933 | C | 15 | ATM | |
| CCT129202 | C | 16 | Aurora Kinase | Cell Cycle |
| LY294002 | C | 17 | PI3K | |
| R406 (free base) | C | 18 | Syk | Angiogenesis |
| JNJ-38877605 | C | 19 | c-Met | |
| KU-60019 | C | 20 | ATM | DNA Damage |
| Brivanib | C | 21 | VEGFR, FGFR | |
| alaninate (BMS- | | | | |
| 582664) | | | | |
| KW 2449 | C | 22 | Flt, Bcr-Abl, | Angiogenesis |
| | | | Aurora Kinase | |
| Raf265 | D | 3 | VEGFR, Rat | |
| derivative | | | | |
| NVP-BVU972 | D | 4 | c-Met | Protein Tyrosine |
| | | | | Kinase |
| PP242 | D | 5 | mTOR | PI3K/Akt/mTOR |
| TAK-285 | D | 6 | EGFR, HER2 | Protein Tyrosine |
| | | | | Kinase |
| Palomid 529 | D | 7 | mTOR, PI3K | |
| GDC-0068 | D | 8 | Akt | PI3K/Akt/mTOR |
| TAK-733 | D | 9 | MEK | MAPK |
| Desmethyl | D | 10 | EGFR | |
| Erlotinib (CP- | | | | |
| 473420) | | | | |
| DCC-2036 | D | 11 | Bcr-Abl | Angiogenesis |
| (Rebastinib) | | | | |
| TG 100713 | D | 12 | PI3K | PI3K/Akt/mTOR |
| AS-252424 | D | 13 | PI3K | PI3K/Akt/mTOR |
| Bosutinib (SKI- | D | 14 | Src | Angiogenesis |
| 606) | | | | |
| NVP-BSK805 | D | 15 | JAK | JAK/STAT |
| SNS-314 | D | 16 | Aurora Kinase | |
| Mesylate | | | | |
| AMG 900 | D | 17 | Aurora Kinase | Cell Cycle |
| Doxercalciferol | D | 18 | | |
| (Hectorol) | | | | |
| CX-4945 | D | 20 | PKC | |
| (Silmitasertib) | | | | |
| AZ628 | D | 21 | Rat | MAPK |
| Saracatinib | E | 3 | Src, Bcr-Abl | Angiogenesis |
| (AZD0530) | | | | |
| TGX-221 | E | 4 | PI3K | PI3K/Akt/mTOR |
| CI-1033 | E | 5 | EGFR, HER2 | |
| (Canertinib) | | | | |
| PIK-90 | E | 6 | PI3K | |
| Motesanib | E | 7 | VEGFR, | Protein Tyrosine |
| Diphosphate | | | PDGFR, c-Kit | Kinase |
| (AMG-706) | | | | |
| JNJ-7706621 | E | 8 | CDK, Aurora | Cell Cycle |
| | | | Kinase | |
| Sunitinib Malate | E | 9 | VEGFR, | Microbiology |
| (Sutent) | | | PDGFR, c-Kit, Flt | |
| TG100-115 | E | 10 | PI3K | PI3K/Akt/mTOR |
| Masitinib | E | 11 | c-Kit, PDGFR, | |
| (AB1010) | | | FGFR, FAK | |
| Staurosporine | E | 12 | PKC | TGF-beta/Smad |
| SB 202190 | E | 13 | p38 MAPK | PI3K/Akt/mTOR |
| SB 525334 | E | 14 | TGF-beta/Smad | TGF-beta/Smad |
| GSK1904529A | E | 15 | IGF-1R | |
| XL765 | E | 16 | PI3K, mTOR | PI3K/Akt/mTOR |
| OSU-03012 | E | 17 | PDK-1 | |
| CP 673451 | E | 18 | | Protein Tyrosine |
| | | | | Kinase |
| PD 0332991 | E | 19 | CDK | |
| (Palbociclib) HCl | | | | |
| BS-181 HCl | E | 20 | CDK | Cell Cycle |
| AG-490 | E | 21 | JAK, EGFR | |
| BMS 794833 | F | 3 | c-Met, VEGFR | Protein Tyrosine |
| | | | | Kinase |
| CH5424802 | F | 4 | ALK | |
| Cyt387 | F | 5 | JAK | JAK/STAT |
| INCB28060 | F | 6 | c-Met | |
| WP1130 | F | 7 | DUB, Bcr-Abl | Angiogenesis |
| INK 128 | F | 8 | mTOR | PI3K/Akt/mTOR |
| (MLN0128) | | | | |
| LDN193189 | F | 9 | TGF-beta/Smad | |
| Torin 1 | F | 10 | Autophagy, | |
| | | | mTOR | |
| CCT128930 | F | 11 | Akt | PI3K/Akt/mTOR |
| Piceatannol | F | 12 | | Angiogenesis |
| PF-00562271 | F | 13 | FAK | Angiogenesis |
| Motesanib | F | 14 | VEGFR, | Protein Tyrosine |
| Diphosphate | | | PDGFR, c-Kit | Kinase |
| (AMG-706) | | | | |
| WAY-600 | F | 15 | mTOR | PI3K/Akt/mTOR |
| BIIB021 | F | 16 | HSP | Cytoskeletal |
| | | | | Signaling |
| ZM 336372 | F | 17 | Rat | MAPK |
| XL765 | F | 18 | PI3K, mTOR | PI3K/Akt/mTOR |
| (SAR245409) | | | | |
| TG101348 | F | 19 | JAK | JAK/STAT |
| (SAR302503) | | | | |
| Mitoxantrone HCl | F | 20 | | |
| AMG458 | F | 21 | c-Met | Protein Tyrosine |
| | | | | Kinase |
| AZD6244 | G | 3 | MEK | MAPK |
| (Selumetinib) | | | | |
| WZ3146 | G | 4 | EGFR | Protein Tyrosine |
| | | | | Kinase |
| CI-1040 | G | 5 | MEK | MAPK |
| (PD184352) | | | | |
| PIK-75 | G | 6 | PI3K, DNA-PK | PI3K/Akt/mTOR |
| Nilotinib (AMN- | G | 7 | Bcr-Abl | Angiogenesis |
| 107) | | | | |
| PD173074 | G | 8 | FGFR, VEGFR | Angiogenesis |
| Tandutinib | G | 9 | Flt | |
| (MLN518) | | | | |
| GSK1059615 | G | 10 | PI3K, mTOR | PI3K/Akt/mTOR |
| GDC-0941 | G | 11 | PI3K | Metabolism |
| Aurora A | G | 12 | Aurora Kinase | Cell Cycle |
| Inhibitor I | | | | |
| MK-2206 2HCl | G | 13 | Akt | |
| HMN-214 | G | 14 | PLK | Cell Cycle |
| PF-04217903 | G | 15 | c-Met | |
| AT7519 | G | 16 | CDK | Cell Cycle |
| Danusertib | G | 17 | Aurora Kinase, | |
| (PHA-739358) | | | FGFR, Bcr-Abl, | |
| | | | c-RET, Src | |
| AZD8055 | G | 18 | mTOR | PI3K/Akt/mTOR |
| Triciribine | G | 19 | Akt | |
| (Triciribine | | | | |
| phosphate) | | | | |
| BIRB 796 | G | 20 | p38 MAPK | MAPK |
| (Doramapimod) | | | | |
| SNS-032 (BMS- | G | 21 | CDK | |
| 387032) | | | | |
| LY2784544 | G | 22 | JAK | JAK/STAT |
| NVP-BHG712 | H | 3 | VEGFR, Src, | Protein Tyrosine |
| | | | Raf, Bcr-Abl | Kinase |
| 3-Methyladenine | H | 4 | PI3K | PI3K/Akt/mTOR |
| SB590885 | H | 5 | Raf | MAPK |
| Tofacitinib (CP- | H | 6 | JAK | JAK/STAT |
| 690550, | | | | |
| Tasocitinib) | | | | |
| BKM120 (NVP- | H | 7 | PI3K | PI3K/Akt/mTOR |
| BKM120) | | | | |
| BYL719 | H | 8 | PI3K | PI3K/Akt/mTOR |
| AZD5438 | H | 9 | CDK | Cell Cycle |
| SAR131675 | H | 10 | VEGFR | |
| A66 | H | 11 | PI3K | PI3K/Akt/mTOR |
| Tofacitinib citrate | H | 12 | JAK | JAK/STAT |
| (CP-690550 | | | | |
| citrate) | | | | |
| GSK1120212 | H | 13 | MEK | MAPK |
| (Trametinib) | | | | |
| Vandetanib | H | 14 | VEGFR | Endocrinology & |
| (Zactima) | | | | Hormones |
| TG101209 | H | 15 | Flt, JAK, c-RET | JAK/STAT |
| Thalidomide | H | 16 | | Apoptosis |
| PF-03814735 | H | 17 | Aurora Kinase | |
| BMS 777607 | H | 18 | c-Met | Protein Tyrosine |
| | | | | Kinase |
| PKI-402 | H | 19 | PI3K | |
| DCC-2036 | H | 20 | Bcr-Abl | Angiogenesis |
| (Rebastinib) | | | | |
| NVP-BGT226 | H | 21 | PI3K, mTOR | PI3K/Akt/mTOR |
| BEZ235 (NVP- | I | 3 | ATM/ATR, | |
| BEZ235) | | | mTOR, PI3K | |
| CYC116 | I | 4 | Aurora Kinase, | Cell Cycle |
| | | | VEGFR | |
| Dasatinib (BMS- | I | 5 | Src, Bcr-Abl, c- | Angiogenesis |
| 354825) | | | Kit | |
| Tivozanib (AV- | I | 6 | VEGFR, c-Kit, | Protein Tyrosine |
| 951) | | | PDGFR | Kinase |
| Pazopanib HCl | I | 7 | VEGFR, | Protein Tyrosine |
| | | | PDGFR, c-Kit | Kinase |
| WYE-354 | I | 8 | mTOR | PI3K/Akt/mTOR |
| Temsirolimus | I | 9 | mTOR | Neuronal |
| (Torisel) | | | | Signaling |
| MGCD-265 | I | 10 | c-Met, VEGFR, | Protein Tyrosine |
| | | | Tie-2 | Kinase |
| SB 431542 | I | 11 | TGF-beta/Smad | |
| PHA-680632 | I | 12 | Aurora Kinase | Cell Cycle |
| PD153035 HCl | I | 13 | EGFR | |
| AEE788 (NVP- | I | 14 | EGFR, Flt, | Protein Tyrosine |
| AEE788) | | | VEGFR, HER2 | Kinase |
| MLN8054 | I | 15 | Aurora Kinase | |
| Quizartinib | I | 16 | Flt | Angiogenesis |
| (AC220) | | | | |
| TAE684 (NVP- | I | 17 | ALK | |
| TAE684) | | | | |
| PHT-427 | I | 18 | Akt, PDK-1 | PI3K/Akt/mTOR |
| XL147 | I | 19 | PI3K | |
| Tie2 kinase | I | 20 | Tie-2 | Protein Tyrosine |
| inhibitor | | | | Kinase |
| Barasertib | I | 21 | Aurora Kinase | |
| (AZD1152- | | | | |
| HQPA) | | | | |
| BGJ398 (NVP- | I | 22 | FGFR | |
| BGJ398) | | | | |
| OSI-420 | J | 3 | EGFR | Protein Tyrosine |
| | | | | Kinase |
| Dinaciclib | J | 4 | CDK | Cell Cycle |
| (SCH727965) | | | | |
| Apatinib | J | 5 | VEGFR | Protein Tyrosine |
| (YN968D1) | | | | Kinase |
| Sotrastaurin | J | 6 | PKC | TGF-beta/Smad |
| (AEB071) | | | | |
| CX-4945 | J | 7 | PKC | |
| (Silmitasertib) | | | | |
| Tyrphostin AG | J | 8 | HER2 | Protein Tyrosine |
| 879 (AG 879) | | | | Kinase |
| PP-121 | J | 9 | DNA-PK, mTOR, | Protein Tyrosine |
| | | | PDGF | Kinase |
| Semaxanib | J | 10 | VEGFR | Protein Tyrosine |
| (SU5416) | | | | Kinase |
| NU7441 (KU- | J | 11 | DNA-PK, PI3K | DNA Damage |
| 57788) | | | | |
| VX-702 | J | 12 | p38 MAPK | MAPK |
| Flavopiridol | J | 13 | CDK | Cell Cycle |
| hydrochloride | | | | |
| Masitinib | J | 14 | c-Kit, PDGFR, | |
| (AB1010) | | | FGFR, FAK | |
| GDC-0980 | J | 15 | mTOR, PI3K | PI3K/Akt/mTOR |
| (RG7422) | | | | |
| Abitrexate | J | 16 | DHFR | Metabolism |
| (Methotrexate) | | | | |
| PH-797804 | J | 17 | p38 MAPK | MAPK |
| Estrone | J | 18 | | Endocrinology & |
| | | | | Hormones |
| GSK1070916 | J | 19 | Aurora Kinase | |
| CH5132799 | J | 20 | PI3K, mTOR | PI3K/Akt/mTOR |
| PHA-848125 | J | 21 | CDK | Cell Cycle |
| BIBF1120 | K | 3 | VEGFR, | Protein Tyrosine |
| (Vargatef) | | | PDGFR, FGFR | Kinase |
| WZ4002 | K | 4 | EGFR | Protein Tyrosine |
| | | | | Kinase |
| Deforolimus | K | 5 | mTOR | |
| (Ridaforolimus) | | | | |
| YM201636 | K | 6 | PI3K | PI3K/Akt/mTOR |
| PD0325901 | K | 7 | MEK | DNA Damage |
| Vemurafenib | K | 8 | Raf | MAPK |
| (PLX4032) | | | | |
| Vandetanib | K | 9 | VEGFR | Endocrinology & |
| (Zactima) | | | | Hormones |
| ON-01910 | K | 10 | PLK | Cell Cycle |
| Crizotinib (PF- | K | 11 | c-Met, ALK | |
| 02341066) | | | | |
| Thiazovivin | K | 12 | ROCK | Cell Cycle |
| SU11274 | K | 13 | c-Met | Neuronal |
| | | | | Signaling |
| PHA-793887 | K | 14 | CDK | Cell Cycle |
| Vatalanib 2HCl | K | 15 | VEGFR, c-Kit, Flt | |
| (PTK787) | | | | |
| Hesperadin | K | 16 | Aurora Kinase | Cell Cycle |
| BI 2536 | K | 17 | PLK | |
| KRN 633 | K | 18 | VEGFR, PDGFR | Protein Tyrosine |
| | | | | Kinase |
| XL-184 free base | K | 19 | VEGFR, c-Met, | |
| (Cabozantinib) | | | Flt, Tie-2, c-Kit | |
| TWS119 | K | 20 | GSK-3 | PI3K/Akt/mTOR |
| PLX-4720 | K | 21 | Raf | |
| AST-1306 | K | 22 | EGFR | Protein Tyrosine |
| | | | | Kinase |
| R935788 | L | 3 | Syk | |
| (Fostamatinib | | | | |
| disodium, R788 | | | | |
| disodium) | | | | |
| Dovitinib Dilactic | L | 4 | Flt, FGFR, | Angiogenesis |
| acid (TKI258 | | | PDGFR, | |
| Dilactic acid) | | | VEGFR, c-Kit | |
| CAL-101 (GS- | L | 5 | PI3K | PI3K/Akt/mTOR |
| 1101) | | | | |
| WP1066 | L | 6 | JAK | JAK/STAT |
| Indirubin | L | 7 | GSK-3 | PI3K/Akt/mTOR |
| Torin 2 | L | 8 | mTOR | PI3K/Akt/mTOR |
| OSI-027 | L | 9 | mTOR | PI3K/Akt/mTOR |
| Baricitinib | L | 10 | JAK | |
| (LY3009104) | | | | |
| GSK2126458 | L | 11 | PI3K, mTOR | PI3K/Akt/mTOR |
| PCI-32765 | L | 13 | Src | Angiogenesis |
| (Ibrutinib) | | | | |
| SU11274 | L | 14 | c-Met | Neuronal |
| | | | | Signaling |
| A-769662 | L | 15 | AMPK | PI3K/Akt/mTOR |
| Epirubicin HCl | L | 16 | Topoisomerase | |
| Dacomitinib | L | 17 | EGFR | Protein Tyrosine |
| (PF299804, PF- | | | | Kinase |
| 00299804) | | | | |
| Azacitidine | L | 18 | DNA/RNA | DNA Damage |
| (Vidaza) | | | Synthesis | |
| PHA-767491 | L | 19 | CDK | Cell Cycle |
| TG101348 | L | 20 | JAK | JAK/STAT |
| (SAR302503) | | | | |
| Arry-380 | L | 21 | HER2 | |
| Afatinib | M | 3 | EGFR | Protein Tyrosine |
| (BIBW2992) | | | | Kinase |
| PD98059 | M | 4 | MEK | MAPK |
| Erlotinib HCl | M | 5 | EGFR | Protein Tyrosine |
| | | | | Kinase |
| OSI-930 | M | 6 | c-Kit, VEGFR | Protein Tyrosine |
| | | | | Kinase |
| IC-87114 | M | 8 | PI3K | |
| VX-680 (MK- | M | 9 | Aurora Kinase | Endocrinology & |
| 0457, | | | | Hormones |
| Tozasertib) | | | | |
| Ki8751 | M | 10 | VEGFR, c-Kit, | Protein Tyrosine |
| | | | PDGFR | Kinase |
| PHA-665752 | M | 11 | c-Met | |
| SP600125 | M | 12 | JNK | MAPK |
| Brivanib (BMS- | M | 13 | VEGFR, FGFR | GPCR & G |
| 540215) | | | | Protein |
| PIK-93 | M | 14 | PI3K, VEGFR | PI3K/Akt/mTOR |
| U0126-EtOH | M | 15 | MEK | |
| BIX 02188 | M | 16 | MEK | MAPK |
| Foretinib | M | 17 | c-Met, VEGFR | |
| (GSK1363089, | | | | |
| XL880) | | | | |
| AT7867 | M | 18 | Akt, S6 kinase | PI3K/Akt/mTOR |
| Everolimus | M | 19 | mTOR | |
| (RAD001) | | | | |
| BMS-265246 | M | 20 | CDK | Cell Cycle |
| Roscovitine | M | 21 | CDK | |
| (Seliciclib, | | | | |
| CYC202) | | | | |
| AZD8931 | M | 22 | EGFR, HER2 | Protein Tyrosine |
| | | | | Kinase |
| PIK-293 | N | 3 | PI3K | PI3K/Akt/mTOR |
| MK-5108 (VX- | N | 4 | Aurora Kinase | Cell Cycle |
| 689) | | | | |
| PIK-294 | N | 5 | PI3K | PI3K/Akt/mTOR |
| AZD4547 | N | 6 | FGFR | Angiogenesis |
| Quercetin | N | 7 | PI3K, PKC, Src, | Epigenetics |
| (Sophoretin) | | | Sirtuin | |
| NVP-TAE226 | N | 8 | FAK | Angiogenesis |
| R788 | N | 9 | Syk | Angiogenesis |
| (Fostamatinib) | | | | |
| Golvatinib | N | 10 | c-Met, VEGFR | Protein Tyrosine |
| (E7050) | | | | Kinase |
| WYE-125132 | N | 11 | mTOR | PI3K/Akt/mTOR |
| AS-604850 | N | 13 | PI3K | PI3K/Akt/mTOR |
| BTZ043 | N | 14 | | |
| racemate | | | | |
| KX2-391 | N | 15 | Src | Angiogenesis |
| Temozolomide | N | 16 | | Ubiquitin |
| AG-1478 | N | 17 | EGFR | Protein Tyrosine |
| (Tyrphostin AG- | | | | Kinase |
| 1478) | | | | |
| Sodium butyrate | N | 18 | | |
| PF-04691502 | N | 19 | mTOR, PI3K, Akt | PI3K/Akt/mTOR |
| APO866 (FK866) | N | 20 | | |
| ARQ 197 | N | 21 | c-Met | Protein Tyrosine |
| (Tivantinib) | | | | Kinase |
| Bosutinib (SKI- | O | 3 | Src | Angiogenesis |
| 606) | | | | |
| Regorafenib | O | 4 | c-Kit, Rat, | Protein Tyrosine |
| (BAY 73-4506) | | | VEGFR | Kinase |
| Gefitinib (Iressa) | O | 5 | EGFR | Protein Tyrosine |
| | | | | Kinase |
| Ku-0063794 | O | 6 | mTOR | PI3K/Akt/mTOR |
| PI-103 | O | 7 | DNA-PK, PI3K, | Neuronal |
| | | | mTOR | Signaling |
| BX-795 | O | 8 | PDK-1, IKK | PI3K/Akt/mTOR |
| Y-27632 2HCl | O | 9 | ROCK | |
| Ruxolitinib | O | 10 | JAK | JAK/STAT |
| (INCB018424) | | | | |
| ZSTK474 | O | 11 | PI3K | Neuronal |
| | | | | Signaling |
| NVP-ADW742 | O | 13 | IGF-1R | |
| Ponatinib | O | 14 | Bcr-Abl, VEGFR, | Angiogenesis |
| (AP24534) | | | FGFR, PDGFR, | |
| | | | Flt | |
| ZM-447439 | O | 15 | Aurora Kinase | |
| BIX 02189 | O | 16 | MEK | MAPK |
| SGX-523 | O | 17 | c-Met | |
| BMS 777607 | O | 18 | c-Met | Protein Tyrosine |
| | | | | Kinase |
| MLN8237 | O | 19 | Aurora Kinase | |
| (Alisertib) | | | | |
| AZD8330 | O | 20 | MEK | MAPK |
| SNS-314 | O | 21 | Aurora Kinase | |
| GSK461364 | O | 22 | PLK | Cell Cycle |
| AZ 960 | P | 3 | JAK | JAK/STAT |
| MK-2461 | P | 4 | c-Met, FGFR, | Protein Tyrosine |
| | | | PDGFR | Kinase |
| Telatinib (BAY | P | 5 | VEGFR, | Protein Tyrosine |
| 57-9352) | | | PDGFR, c-Kit | Kinase |
| CEP33779 | P | 6 | JAK | JAK/STAT |
| Imatinib | P | 7 | PDGFR, c-Kit, v- | Protein Tyrosine |
| (Gleevec) | | | Abl | Kinase |
| Tideglusib | P | 8 | GSK-3 | |
| LY2603618 (IC- | P | 9 | Chk | Cell Cycle |
| 83) | | | | |
| IMD 0354 | P | 10 | IKK | NF-?B |
| WYE-687 | P | 11 | mTOR | |
| CAY10505 | P | 13 | PI3K | |
| PD 0332991 | P | 14 | CDK | |
| (Palbociclib) HCl | | | | |
| GSK1838705A | P | 15 | IGF-1, ALK | Protein Tyrosine |
| | | | | Kinase |
| BX-795 | P | 16 | PDK-1, IKK | PI3K/Akt/mTOR |
| SB 415286 | P | 17 | GSK-3 | PI3K/Akt/mTOR |
| MLN9708 | P | 18 | Proteasome | Proteases |
| CCT137690 | P | 19 | Aurora Kinase | Cell Cycle |
| BAY 11-7082 | P | 20 | I?B/IKK | NF-?B |
| (BAY 11-7821) | | | | |
| ARRY334543 | P | 21 | EGFR | Protein Tyrosine |
| | | | | Kinase |

### EXAMPLES

### Method

### Generation of 3D Tumour Spheroids

Generation of 3D tumour spheroids may be accomplished using different protocols, including the hanging drop technology and ultra-low attachment plates. The typical technique involves reducing cell-surface contact and encouraging cellular aggregation to facilitate cell-cell coupling into spheroids. The method disclosed herein is independent of the techniques used to generate the 3D tumour spheroids. However, the method requires that the pre-treatment spheroids are optimally formed with an average size of between 350 um to 500 um (microns), and presenting well-defined necrotic core, quiescent, and proliferating zones.

The examples used in this disclosure were generated by seeding 5000 cells into each well of Corning® 384 Well Black Clear Round Bottom Ultra-Low Attachment Spheroid Microplates. The assay plates were incubated at 37° C., 5% CO₂ over 3 days to allow formation of the tumour spheroids. At 96 hours, the spheroids were imaged (labelled as “untreated” in the study) using a confocal microscope at 20× (Perkin Elmer Opera Phenix High Content Screening system) and then treated at 1 μM of the compounds (in DMSO). In total, 1,231 spheroids were generated and treated in duplicates at 96 hours with small molecule and kinase inhibitors from the Selleck Anti-cancer library and Selleck Kinase Inhibitor chemical library. The drug treated spheroids were imaged subsequently at 24, 48 and 72 hours after treatment.

### Computational Segmentation of 3D Tumour Spheroids

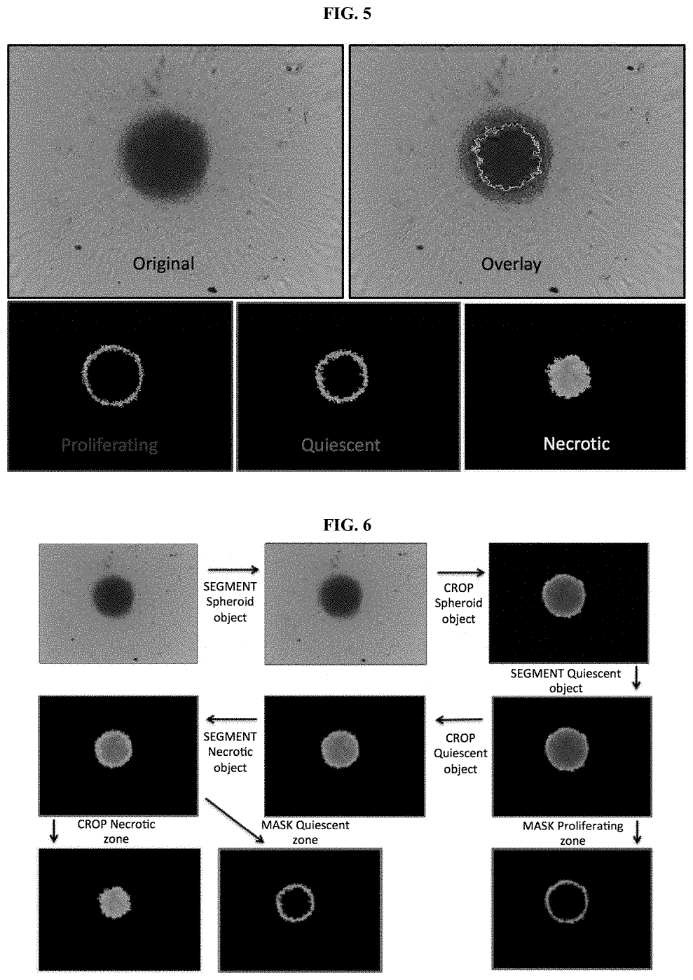

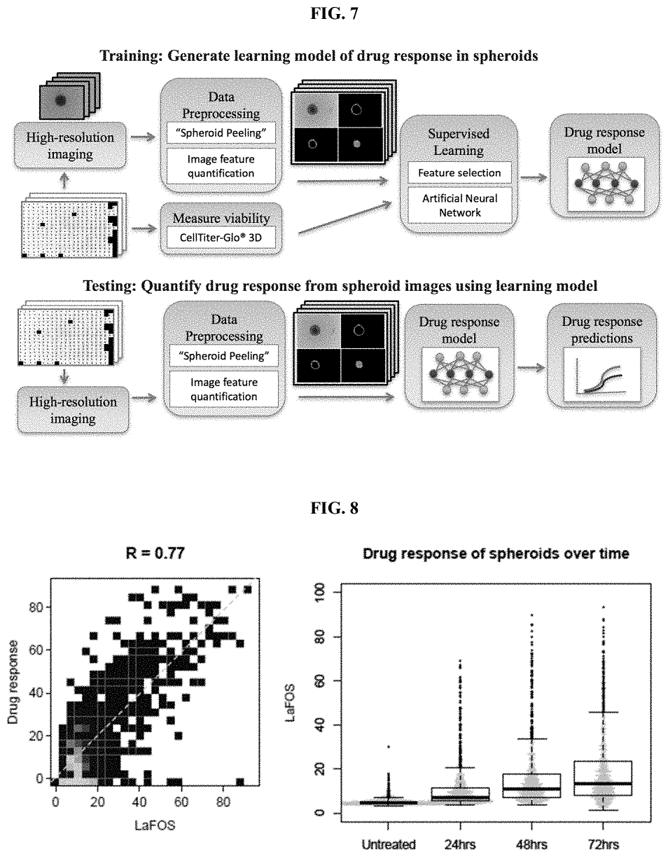

Bright-field images of the 1,231 spheroids acquired at different time-points and z-planes were computationally segmented into proliferating (red), quiescent (green) and necrotic (yellow) zones through a method referred to as “Spheroid Peeling” (FIG. 5). Other imaging methods can also be used—Phase contrast imaging, electron microscopy, wide-field, dark-field, super-resolution microscopy, and many more.

Briefly, “Spheroid Peeling” involves repeatedly segmenting the spheroid image from the periphery to the core zone. The entire spheroid was first segmented as an object (hereby referred to as spheroid object) and cropped from the original well image (FIG. 6). An “inner core” was then identified from the spheroid object, hereby referred to as quiescent object. The proliferating zone was obtained by masking the quiescent object from the spheroid object. Similarly, the necrotic zone was identified as the “inner core” of the quiescent object, and the quiescent zone was obtained by masking the necrotic zone from the quiescent object. A number of image features were quantified from each of the proliferating, quiescent and necrotic zones, resulting in a total of 504 image descriptors for each spheroid. The “spheroid peeling” algorithm can be written in a number of languages or image analysis tool, such as Cell Profiler, MATLAB or ImageJ.

### Modeling Drug Response Using Multi-Variate Learning-Based Method

As shown in FIG. 4, individual image features are of limited predictive value to drug response at R<0.5. However, the accuracy can be substantially improved by generating a multivariate image feature model using learning-based methods. The method used in the present example is based on Artificial Neural Network (ANN)/Deep Learning, although any machine learning approach (e.g Support Vector Machine, Random Forest, Regression) can be used to build the drug response prediction model. A non-exhaustive listing of learning based methods that can be used in accordance with the present disclosure to generate the multivariate image feature model include, but is not limited to, Artificial Neural Networks (ANN), Deep Learning (such as Convolutional Neural Networks), Support Vector Machines (SVM), Regression-based approaches (such as linear regression or logistic regression), Tree-based approaches (such as Decision Tree or Random Forest approaches), Boosting Approaches (such as Gradient Boost or Adaboost approaches), Distance-based approaches (such as K-nearest neighbours (i.e. KNN) or K-means approaches) and dimension reduction algorithms (such as Principal Component Analysis (PCA)).

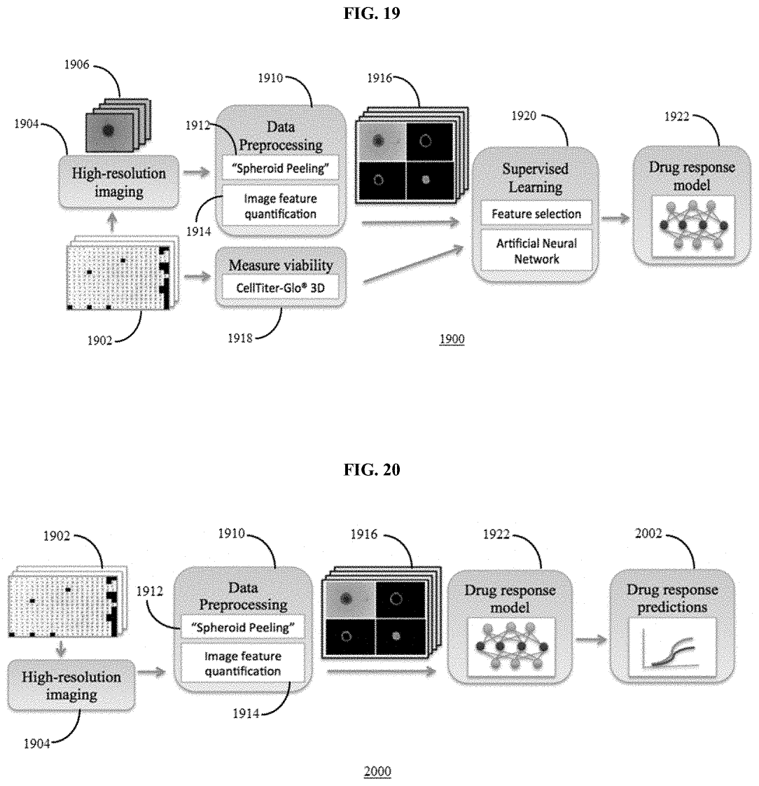

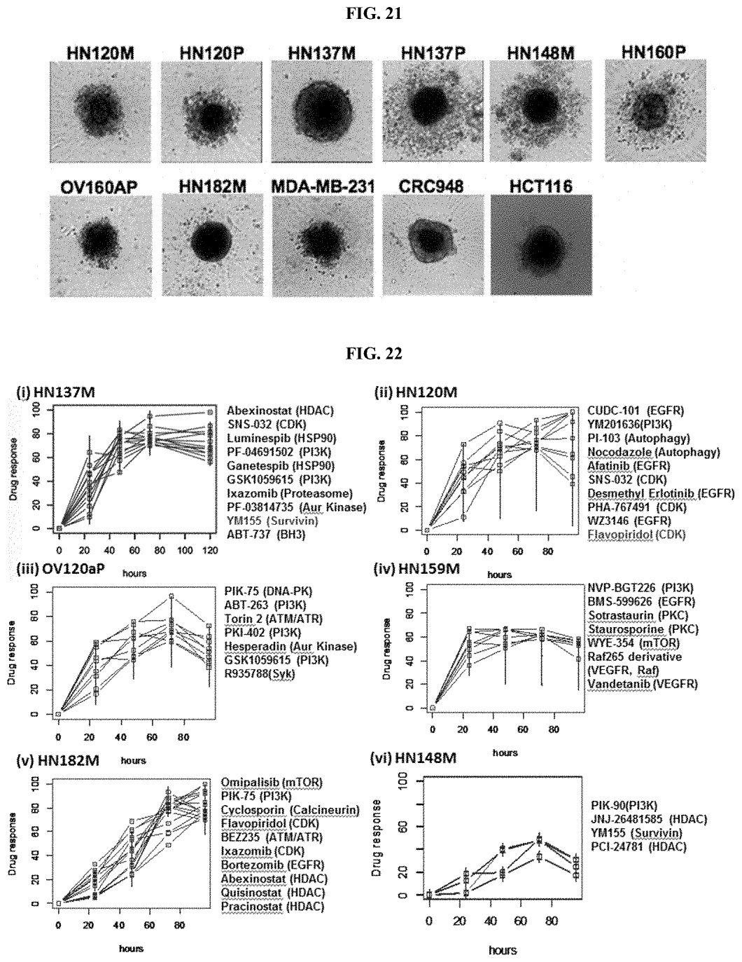

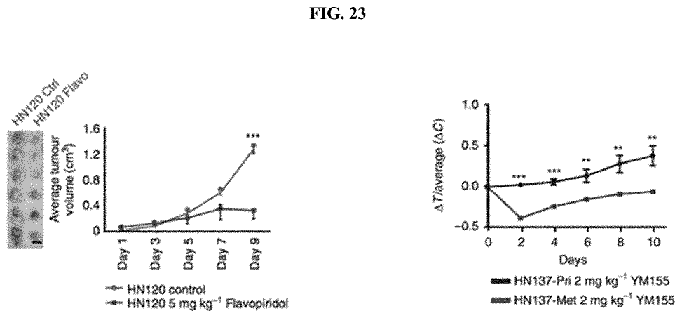

The overall workflow of the method is shown in FIG. 19. FIG. 19 is a flow diagram 700 showing a machine learning based training method in accordance with the present disclosure for generating a computational model (or learning model) of drug response in spheroids. Two sets of spheroids were generated for imaging and for viability measurement, respectively. The two sets of spheroids were treated with the same drugs. For feasibility testing, a training set of 1,231 spheroids 1902 was generated. At 72 hours after drug treatment, acquisition 1904 of bright-field images 1906 was obtained. As data preprocessing 708, “Spheroid Peeling” 1910 was applied to obtain, through image feature quantification 1912, 504 image descriptors from the Proliferating, Quiescent and Necrotic zones of each tumour spheroid, resulting in over ˜600K image descriptors 1916 in total.

In parallel, a duplicate set of 1,231 spheroids was cultured and the viability of each spheroid in the presence of drug treatment was measured 1918 using an end-point CellTiter-Glo® 3D Cell Viability Assay (72 hours). An inhibition score was calculated for each tumour spheroid by normalizing the ATP readouts (in RLU) to that of the DMSO wells of each plate. The image features and corresponding inhibition scores of each spheroid were used as input to supervised learning 1920.

The learning process generated a computational model 1922 that can be perceived as a complex multi-feature numerical quantification of the drug response of the spheroids. Generating the computational model includes one or more of training the computational model or determining parameters of the computational model. The scores predicted from this learning model are referred to as LaFOS (Label Free Oncology Score) and can be considered an inhibition score of the test agent with respect to the 3D cell structure or an activity (or response) score of the test agent with respect to a 3D cell structure. This model can be used to predict drug activity on spheroids cultured from the same patient or different patients.

Referring to FIG. 20, a drug-testing platform in accordance with the present disclosure can effectively include a one-time training step 2000, with the learned model 1922 applied subsequently to images of spheroids derived from the same or other patients at multiple time-points to obtain drug response predictions 2002. However, to avoid morphological variances due to differences in experimental set-up, the tumour spheroids were cultured using similar protocols. In practice, the drug testing workflow simply requires imaging the drug treated spheroids at regular intervals. Morphological changes over time are profiled to determine the response kinetics of different drugs on the spheroids.

In the performed feasibility test, each of the 1,231 spheroids was imaged at 4 time-points (untreated, 24, 48 and 72 hours) after treatment with one of the 480 anti-cancer drugs, resulting in 4,924 images in total. These images are used as input at the testing stage to generate a comprehensive profile of the response dynamics of the spheroids in presence of different drugs.

Compared to individual image features (FIG. 4), the LaFOS of the spheroids at 72 hours show a significantly higher correlation with the inhibition scores of the spheroids (R=0.77, RMSE=13.2). When applied to all time-points, the LaFOS of majority of the spheroids increase over time (FIG. 8b). This was as expected and suggests that the LaFOS is capable of capturing the increasing sensitivity of the spheroids to the drug treatments over time.

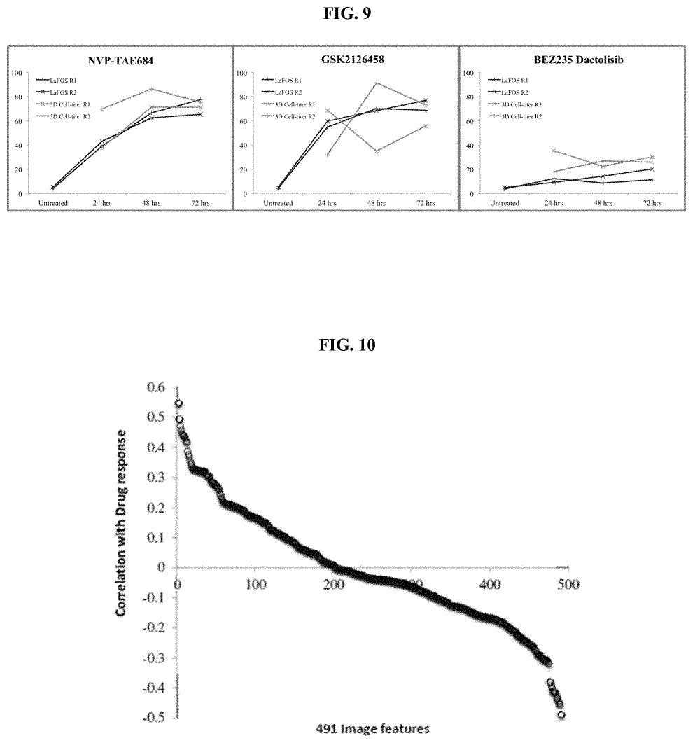

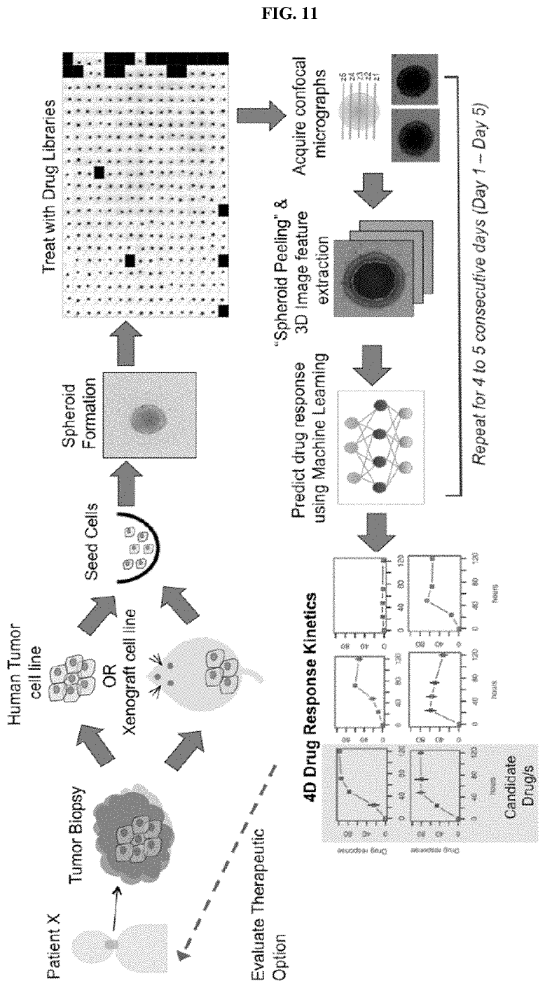

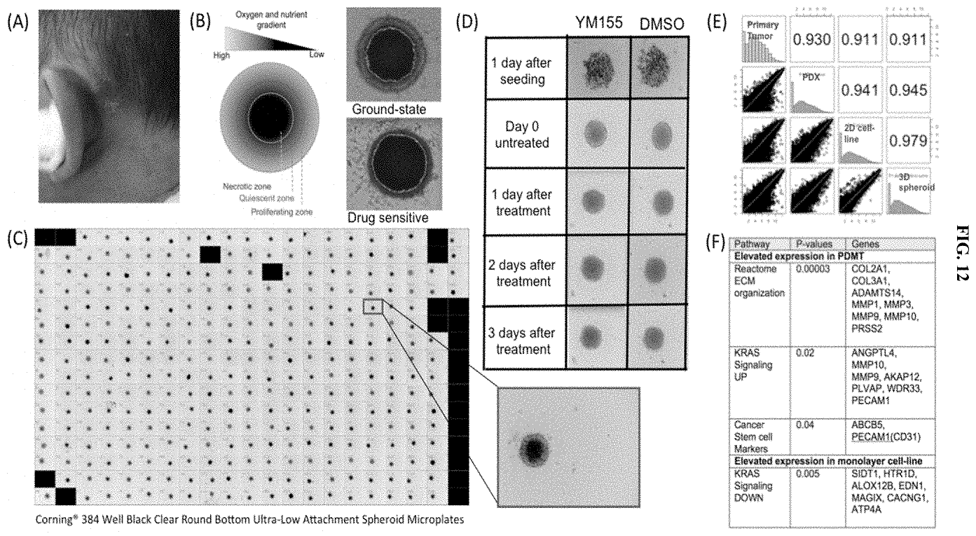

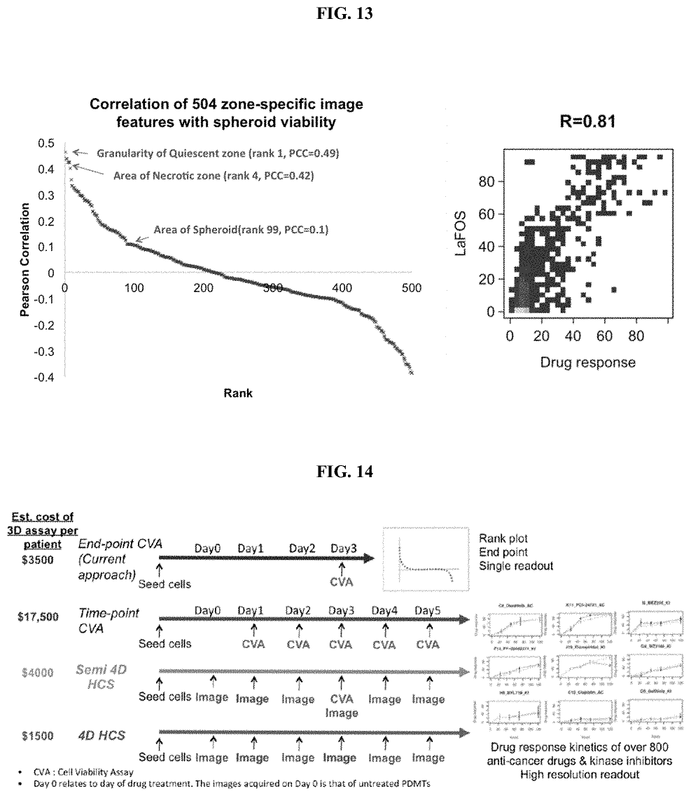

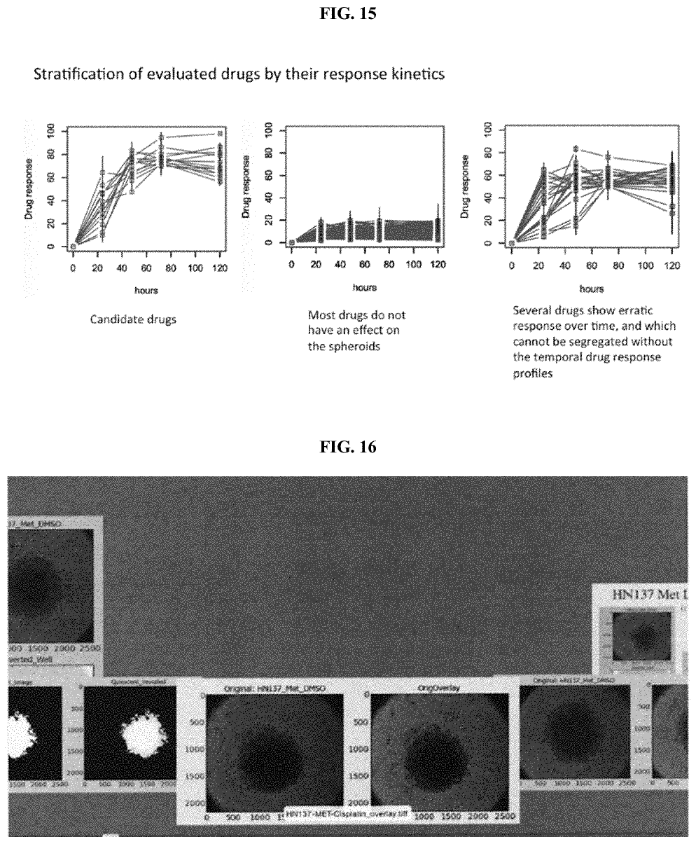

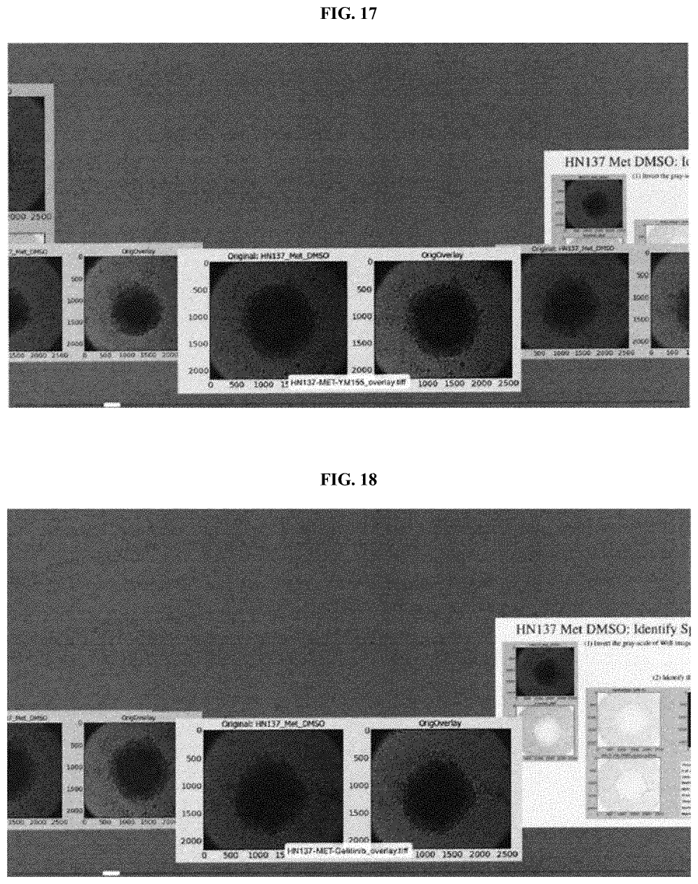

Nevertheless, the time-course profiles show that majority of the compounds does not have an efficacy on the 3D tumour spheroids—the median of LaFOS at 72 hours is less than 20. Only a few compounds show an efficacy of greater than 50% at 72 hours. For instance, the LaFOS remain unchanged for BEZ235 (FIG. 9). Few selected compounds, such as NVP-TAW684 and GSK2126458 show increasing efficacy on the tumour spheroids over the course of 3 days after drug treatment, indicating that the method can be used to profile the pharmacokinetics of the drugs on the 3D tumour spheroids.

Generation and passaging of PDXs. Tumour samples were obtained from patients post-surgery after obtaining informed patient consent in accordance to SingHealth Centralized Institutional Review Board (CIRB: 2014/2093/B). Tumours were minced into 1 mm³ fragments and suspended in a mixture of 5% Matrigel (Corning, cat. no. 354234) in DMEM/F12 (Thermo Fisher, cat. no. 10565-018). The tumour fragment mixtures were then implanted subcutaneously into the left and right flanks of 5-7 weeks old NSG (NOD.Cg-Prkdcscid II2rgtm1Wjl/SzJ) (Jackson Laboratory, stock no. 005557) mice, using 18-gauge needles. Tumours were excised and passaged when they reached 1.5 cm³. For passaging, tissues were cut into small fragment of 1 mm³ prior to resuspension in 20% Matrigel/DMEM/F12 mix, before subcutaneous inoculation of tumour fragments into 5-7 weeks old NSG mice. Protocols for all the animal experiments described were approved by the A*STAR Biological Resource Centre (BRC) Institutional Animal Care and Use Committee (IACUC) under protocol #151065.

### Derivation of PDC Cell Lines and Cell Culture.

Tumours were minced prior to enzymatic dissociation using 4mgmL.1 collagenase type IV (Thermo Fisher, cat. no. 17104019) in DMEM/F12, at 37° C. for 2 h. Cells were washed using cyclical treatment of pelleting and resuspension in phosphate-buffered saline (Thermo Fisher, cat. no 14190235) for three cycles. The final cell suspensions were strained through 70 μm cell strainers (Falcon, cat. no. 352350), prior to pelleting and resuspension in RPMI (Thermo Fisher, cat. no 61870036), supplemented with 10% foetal bovine serum (Biowest, cat. no. S181B) and 1% penicillin-streptomycin (Thermo Fisher, cat. no. 15140122). Cells were kept in a humidified atmosphere of 5% CO₂ at 37° C. Cell line identity was authenticated by comparing the STR profile (Indexx BioResearch) of each cell line to its original tumour. Cells were routinely screened for mycoplasma contamination using Venor® GEM OneStep mycoplasma detection kit (Minerva Biolabs, cat. no. 11-8100).

### Drug Preparation and In Vivo Treatment.

Gefitinib (Iressa) was prepared by dissolving a 250 mg clinical grade tablet (AstraZeneca) in sterile water containing 0.05% Tween-80 (Sigma-Aldrich, cat. no. P4780) to a concentration of 10 mg/mL and administered at a dosage of 25 mg/kg daily via oral gavage. YM155 (Selleckchem, cat. no. S1130) was dissolved in saline to a concentration of 0.5 mg mL/1 and administered by intraperitoneal (i.p.) injection, once every 2 days at 2 mg/kg. Flavopiridol (LC Laboratory, cat. no. A-3499) was dissolved in DMSO to a concentration of 200 mg/mL before diluting to 5 mg/mL using saline and administered by i.p. injection, once every 2 days at 5 mg/kg. Belinostat (Med-Chem Express, cat. no. HY-10225) was dissolved in DMSO to a concentration of 100 mg/mL before diluting to 5 mg/mL using solvent containing (2% Tween-80 and 1% DMSO in saline), and administered at a dosage of 40 mg/kg daily via i.p. injection. Docetaxol was prepared in accordance to published formulation and administered by i.p. injection, once every 2 days at 8 mg/kg. Olaparib was solubilized in DMSO and diluted to 5mgmL-1 with saline containing 10% (w/v) 2-hydroxy-propyl-beta-cyclodextrin (Sigma, cat. no. 332607), and administered at 50 mg/kg daily via i.p. erlotinib was dissolved in 6% captisol (CyDex, Inc., Lenexa, Kans.) in water, pH 4.5 and administered at 150 mg/kg daily via i.p. Control groups for all compounds were treated in their corresponding diluent in the absence of compounds. PDXs were generated by grafting tumours either on both flanks or singly as stated. The length and width of tumours were measured by caliper once every 2 days. Tumour volumes were estimated using the following modified ellipsoidal formula: Tumour volume=½(length×width²). Mice were euthanized when tumours in the control group reaches 2.0 cm³. The weight of tumour was not directly measured, but were estimated using volume where the density of tissue was assumed to be 1 g/cm³. The ratio of the change in treated tumour volume (ΔT) to the average change in control tumour volume (ΔT/Average ΔC) at each time point was calculated as follows:

T=Tumour volume of treatment group

ΔT=Tumour volume of drug-treated group on study day-Tumour volume on initial day of dosing

C=Tumour volume of control group

ΔC=Tumour volume of control group on study day-Tumour volume on initial day of dosing

Average ΔC=Average change in tumour volume across the control-treated group.

1. A method of providing a computational model for predicting an activity of a test agent with respect to a 3D cell structure, the method comprising:
providing a plurality of training samples, each training sample comprising a respective training test agent of a plurality of training test agents applied to a respective training 3D cell structure of a plurality of training 3D cell structures;
determining a respective image of at least one training sample of the plurality of training samples;
determining a respective set of features of each of the respective images;
determining a respective activity of the respective training test agent applied to the respective training 3D cell structure corresponding to at least one training sample of the plurality of training samples; and,
determining the computational model based on the determined respective sets of features and the determined respective activities.

2. The method of claim 1, wherein the respective image of at least one training sample of the plurality of training samples is determined using at least one of bright-field microscopy, phase-contrast microscopy, wide-field microscopy, dark-field microscopy, and super-resolution microscopy.

3. The method of claim 1, wherein determining the respective set of features of each of the respective images comprises processing each of the respective images.

4. The method of claim 3, wherein processing each of the respective images comprises segmenting the image into a plurality of segments, each segment corresponding to a different zone of the training 3D cell structure.

5. The method of claim 4, wherein processing each of the respective images further comprises cropping the plurality of segments to obtain a plurality of zone images.

6. The method of claim 4, wherein the zones comprise a necrotic zone, a quiescent zone, and a proliferating zone.

7. (canceled)

8. The method of claim 4, wherein the respective set of features of each of the respective images are determined based on the segments of the respective image.

9. The method of claim 1, wherein the training 3D cell structure comprises at least one of a spheroid, an organoid, and a tumorsphere.

10. (canceled)

11. The method of claim 4, wherein each set of features is related to at least one of a size or an area or a volume of one of the zones of the respective training 3D cell structure, a curvature of one of the zones of the respective training 3D cell structure, a shape of at least one of the zones of the respective training 3D cell structure, an intensity of at least one of the zones of the respective training 3D cell structure, or a texture of the cells of at least one of the zones of the respective training 3D cell structure.

12. (canceled)

13. The method of claim 1, wherein the computational model is configured to output an activity (or response) score of a test agent with respect to a 3D cell structure.

14.-15. (canceled)

16. The method of claim 1, wherein the computational model comprises at least one machine learning algorithm, including but not limited to an Artificial Neural Network (ANN), Deep Learning (such as but not limited to a Convolutional Neural Network), Support Vector Machine (SVM), Regression-based approaches (such as but not limited to linear regression, logistic regression, and the like), Tree-based approaches (such as but not limited to Decision Tree, Random Forest, and the like), Boosting Approaches (such as but is not limited to Gradient Boost, Adaboost, and the like), Distance-based approaches (such as but is not limited to K-nearest neighbors (i.e. KNN), K-means, and the like), dimension reduction algorithm (such as Principal Component Analysis (PCA), and the like).

17.-21. (canceled)

22. A label-free prediction method comprising:
providing a computational model;
providing a sample comprising a test agent applied to a 3D cell structure;
determining an image of the sample;
determining a set of features of the image; and,
predicting an activity of the test agent with respect to the 3D cell structure based on the set of features and based on the computational model.

23. The prediction method of claim 22, wherein the image of the sample is determined using at least one of bright-field microscopy, phase-contrast microscopy, wide-field microscopy, dark-field microscopy, and super-resolution microscopy.

24. (canceled)

25. The prediction method of claim 23, wherein processing the image comprises segmenting the image into a plurality of segments, each segment corresponding to a different zone of the 3D cell structure.

26. The prediction method of claim 25, wherein processing the image further comprises cropping the plurality of segments to obtain a plurality of zone images.

27.-30. (canceled)

31. The prediction method of claim 22, wherein the set of features is related to at least one of a feature of the 3D cell structure, a feature of a zone of the 3D cell structure, a feature of at least one cell corresponding to the 3D cell structure, or a feature of at least one cell of one of the zones of the 3D cell structure.

32. The prediction method of claim 22, wherein the set of features is related to at least one of a size or an area of one of the zones of the 3D cell structure, a curvature of one of the zones of the 3D cell structure, a shape of at least one of the zones of the 3D cell structure, an intensity of at least one of the zones of the 3D cell structure, or a texture of the cells of at least one of the zones of the 3D cell structure.

33. The prediction method of claim 22, wherein the computational model is configured to output an inhibition score of the test agent with respect to the 3D cell structure.

34. The prediction method of claim 22, wherein the computational model comprises at least one of an Artificial Neural Network (ANN), Deep Learning, Support Vector Machine (SVM), Random Forest, and Regression.

35. The prediction method of claim 22, wherein the computational model comprises the computational model determined according to a method for predicting an activity of a test agent with respect to a 3D structure comprising:
providing a plurality of training samples, each training sample comprising a respective training test agent of a plurality of training test agents applied to a respective training 3D cell structure of a plurality of training 3D cell structures;
determining a respective image of at least one training sample of the plurality of training samples;
determining a respective set of features of each of the respective images;
determining a respective activity of the respective training test agent applied to the respective training 3D cell structure corresponding to at least one training sample of the plurality of training samples; and,
determining the computational model based on the determined respective sets of features and the determined respective activities.

36.-41. (canceled)
